(19) United States
(12) Patent Application Publication
(10) Pub. No.: US 2018/0330814 A1
(43) Pub. Date: Nov. 15, 2018

(54) SYSTEM AND METHODS FOR A CARE MANAGEMENT COMPUTING PLATFORM

(71) Applicant: Direct Care Innovations, LLC, Mesa, AZ (US)

(72) Inventors: Josh Auer, Mesa, AZ (US); Matt Dee, Mesa, AZ (US)

(73) Assignee: Direct Care Innovations, LLC, Mesa, AZ (US)

(21) Appl. No.: 15/979,373

(22) Filed: May 14, 2018

Related U.S. Application Data
(60) Provisional application No. 62/505,768, filed on May 12, 2017.

(51) Int. Cl. G16H 40/20; G16H 10/60

(52) U.S. Cl. CPC G16H 40/20; G16H 10/60

(57) ABSTRACT

Embodiments of a computing platform for care management are disclosed.

### CROSS REFERENCE TO RELATED APPLICATIONS

This non-provisional application claims the benefit of U.S. provisional application Ser. No. 62/505,768 filed on May 12, 2017 which is herein incorporated by reference in its entirety.

### TECHNICAL FIELD

Aspects of the present disclosure relate to care management; and more particularly, to systems and methods for a care management computing platform including one or more modules for generating accounts, wherein the accounts define relationships and rules associated with core entities involved with healthcare management and billing, among other features.

### BACKGROUND

Long term care generally includes direct care, in-home hospice care, behavioral health care, and the like. Billing or funding sources for long term care may include private and public or government sources.

Conventional systems for billing of long term care are inadequate due to the cumbersome and complicated procedures involved with long term care funding sources. As a specific example, services associated with direct care may be billed to and/or funded in part by Medicaid. However, Medicaid billing may involve a specific set of service billing codes and processes in order to process different bills. As another example, rules associated with billing of private and public sources may vary from state to state making it technologically challenging to implement and maintain computing systems that can effectively manage care procedures and billing across state lines.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawings(s) will be provided by the Office upon request and payment of the necessary fee.

FIG. 1A is a system diagram of a care management computing platform, according to aspects of the present disclosure.

FIG. 1B is an entity relationship diagram for the care management computing platform, according to aspects of the present disclosure.

FIG. 1C depicts possible objects of the care management computing platform, according to aspects of the present disclosure.

FIG. 2 is a diagram depicting various possible modules of the care management computing platform, according to aspects of the present disclosure.

FIG. 3 is a process flow diagram illustrating one method for implementing aspects of the care management computing platform, according to aspects of the present disclosure.

FIGS. 4-20 illustrate various screenshots associated with the care management computing platform, according to aspects of the present disclosure.

FIG. 21 illustrates an example of a computing system that may implement various services, systems, and methods of the care management computing platform discussed herein, according to aspects of the present disclosure.

FIGS. 22-25 are screenshots of a mobile application illustrating various novel aspects of the described care management application including electronic visit verification using facial recognition, according to aspects of the present disclosure.

The drawings and other objects, features, and advantages of the present disclosure set forth herein should be apparent from the following description of particular embodiments of those inventive concepts, as illustrated in the accompanying drawings. Also, in the drawings the like reference characters refer to the same parts throughout the different views. The drawings depict only typical embodiments of the present disclosure and, therefore, are not to be considered limiting in scope.

### DETAILED DESCRIPTION

Aspects of the present disclosure involve systems and methods for implementing a computing platform directed to care management (“computing platform”). In particular, in some embodiments the computing platform may include a care management application defining a chart of accounts module which may be used to manage accounts and other modules as described herein. The chart of accounts module may function akin to an accounting system, and include one or more accounts defining relationships between core entities implemented as objects and/or parameters. Core entities may include profiles, time entries, service codes, funding partners, and batches, and the like as described herein. For example, an account of the chart of accounts module may link two profiles together; e.g. define a relationship between a patient and a healthcare provider employee. The chart of accounts module may further define one or more service codes associated with the account that define the scope of work the healthcare provider employee is authorized to provide to the patient. The account of the chart of accounts module may further define funding sources for the account such as Medicaid and/or other public or private sources of funding for healthcare bills generated by the healthcare provider employee. In some embodiments, transactions associated with accounts may be defined using debits and credits, as further described herein.

In some embodiments, the care management application may further include one or more of an authorization module, a training module, a scheduling module, a mobile visit verification module, a payroll time entry module, a billing module, and a reporting module, as described herein. In some embodiments, aspects of any one or more of the modules of the care management application may be implemented by way of an application accessible via a mobile device such as a smartphone.

Aspects of the computing platform may be implemented as a highly scalable, cloud-based software application capable of executing the aforementioned modules stand-alone or as a fully integrated workforce management solution. The modules can be used to increase compliance and eliminate waste and abuse. The computing platform optimizes process flow and compliance for care management with respect to healthcare providers, their employees, patients, and funding sources such as Medicaid.

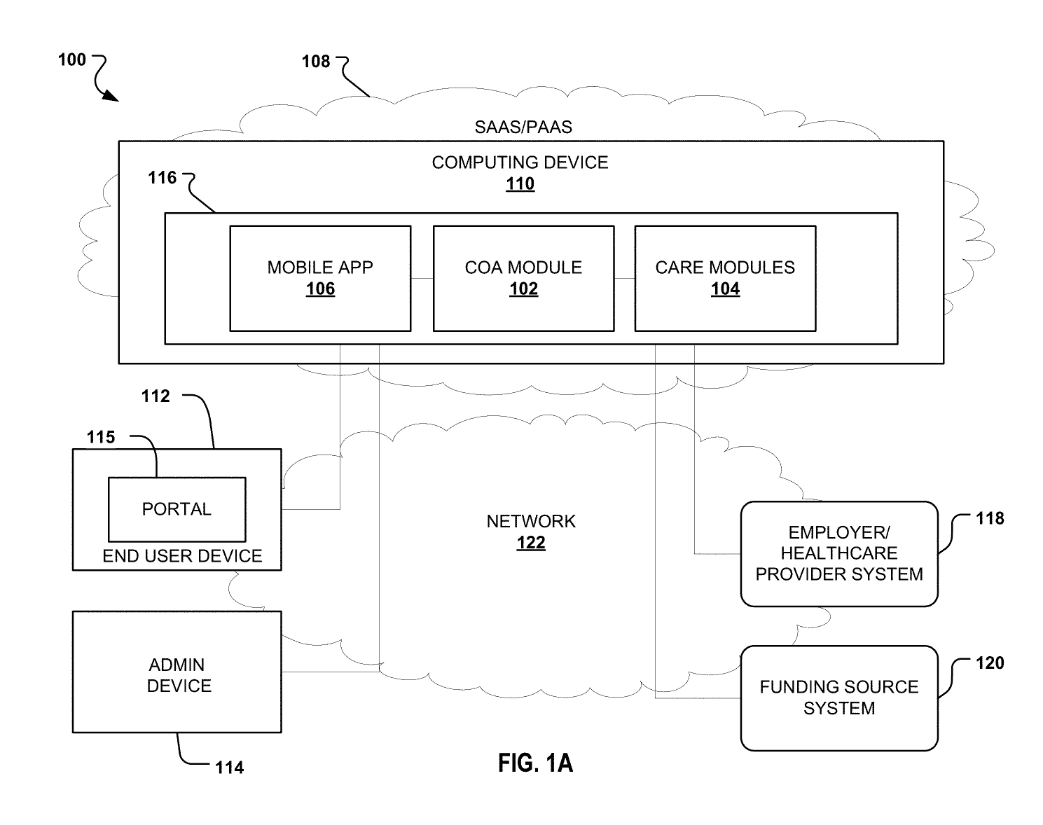

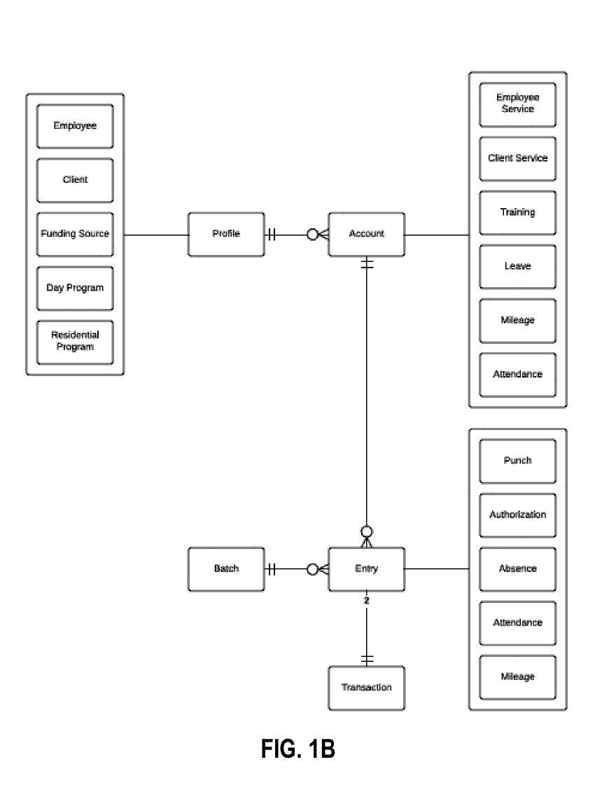

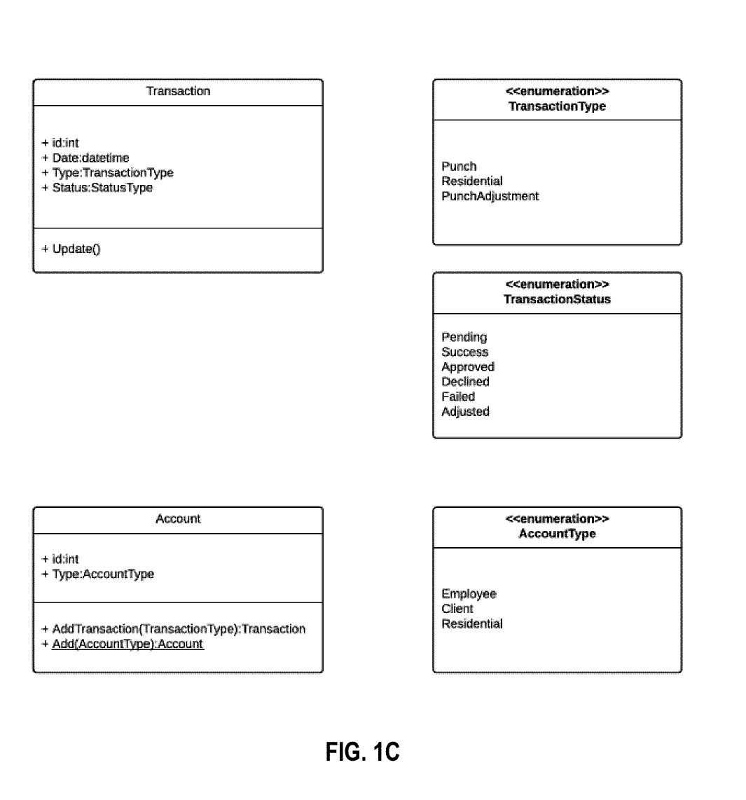

In some embodiments, each of the modules of the care management application may be used independently or integrated with other modules as desired. The modules may be designed to ensure the highest level of compliance possible to government (Medicare, Medicaid, ACA) and private insurance regulations. The computing platform may be configured with program-specific business rule standards, automate compliance at every level, and may be HIPAA, and Sarbanes-Oxley compliant. Additionally, the computing platform may be operable to provide role-based security functionality and be designed for internal business functions as well as external auditing. The computing platform described facilitates the efficient collection of relevant work flow information associated with healthcare services provided to patients, in order to generate accurate, effective, and compliant business outcomes. Referring to the drawings, embodiments of a computing platform for managing patient care are illustrated and generally indicated as 100 in FIGS. 1-25.

### General System Architecture

Referring to FIG. 1A, a computing platform or system architecture directed to healthcare management (“computing platform”), designated 100, is illustrated. In some embodiments, the computing platform 100 may include a computing device 110 for executing aspects of a care management application 116 to provide functionality for end users as described herein. The computing device 110 may comprise at least one device and may comprise a server, rack, mainframe, a desktop computer, terminal, or the like.

In some embodiments, one implementation 108 of the computing device 110 and the care management application 116 may include software as a service (SaaS) such as e.g. cloud-based Microsoft Azure, Amazon Web services, or other such SaaS service. The computing device 110 may also be implemented as part of platform as a service (PaaS). More specifically, by non-limiting example, the computing device 110 may be hosted, implemented, or otherwise by a service provider such as Microsoft that manages the care management application 116 and hardware utilized to implement the care management application 116 (such as the computing device 110) and allows end user devices to connect to and access aspects of the care management application 116 e.g. over the Internet using a web browser.

### Terminology

In the description, terminology is used to describe features of the present inventive concept. For example, references to terms “one embodiment,” “an embodiment,” “the embodiment,” or “embodiments” mean that the feature or features being referred to are included in at least one aspect of the present inventive concept. Separate references to terms “one embodiment,” “an embodiment,” “the embodiment,” or “embodiments” in this description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure as described herein are not essential for its practice.

The term “Portal” refers to a user interface or the like implemented by a browser of a computing device to allow end users to access aspects of the care management application 116.

The term “Company” refers to a core profile of the care management application 116 representing the customer who is paying to license aspects of the care management application 116. This is a special profile of which there can only be per instance of the Portal and it does not support login.

The term “Funding Source” refers to a core profile representing an entity that defines the specifications for service codes and issues authorizations for clients to receive services. Funding Sources may include government entities or divisions (e.g. AZ Dept. of Developmental Disabilities) or private entities (e.g. insurance companies or MCO's).

The term “Cost Center” refers to a department or other unit within an organization to which costs may be charged for accounting purposes.

The term “Employee” refers to a core profile of the care management application 116 representing an end user who provides services to a client or works at a site (e.g. Residential/Day Program). These users can log in and make time punches via the Portal, mobile web, or mobile app. Employee profiles may be assigned roles and/or permissions. In the disability services industry these may also be referred to as a Caregiver, Direct Support Professional (DSP) or Direct Care Worker (DCW).

The term “Supervisor” refers to employee profile which has been assigned the Supervisor role for a cost center.

The term “Employer” refers to an employee profile which has been assigned the Employer role for a cost center.

The term “Client” refers to a core profile of the care management application 116 representing an end user who receives services. In the disability services industry these may also be referred to as a Participant, Consumer, or Patient.

The term “Guardian” refers to a profile of the care management application 116 representing an end user who represents or has legal responsibility for a client. Users of this type can act on behalf of a client for things like requesting or approving services.

The term “Case Worker” refers a profile of the care management application 116 representing a funding source worker. Users of this type can log into the care management application 116 and view a limited subset of information.

The term “Day Program” refers to a profile representing a day program site. Day program sites are unique in that employees can work for them and clients can receive services by being in attendance. For Day Programs an attendance entry must be explicitly created (e.g. no assumption of attendance exists).

The term “Residential Program” refers to a profile representing a residential program site. Residential program sites are unique in that employees can work for them and clients can receive services by being in attendance. For Residential Programs an attendance entry will be created by default unless an absence entry exists for a day (e.g. since clients live at the Residential program the assumption is that they are there unless an absence entry has been created).

The term “Parenting Program” refers to profile representing a parenting program site. Parenting program sites are unique in that employees can work for them and clients can receive services by being in attendance. For Parenting Programs an attendance entry will be created by default unless an absence entry exists for a day (e.g. since clients live at the Parenting program the assumption is that they are there unless an absence entry has been created).

The term “Group Service” refers to a profile representing a group service. Group service profiles allow for an employee to provide services to a group of clients at the same time. The employee is only paid once but the system can bill multiple clients for the same time.

The term “Service Code” refers to a specific service offering as defined by a Funding Source. A Service Code has a specification that defines how it should be provided and billed.

The term “Service Code Statement” refers to a canned question or statement that a company can create and associate with a service code. When a statement is created and linked to a service code they are displayed on the Add New Entry screen along with a check box allowing the end user (employee) to select one or more. A good example would be activity codes that an employee must choose from when making a punch to provide further clarification as to what they were doing during the punch.

The term “Authorization” refers to a special type of entry representing an allotment of dollars/units from a Funding Source to provide a service to a client. An Authorization is for one and only one service code and client. The primary data elements of an Authorization include:
Client
Service Code
Start Date
End Date
Initial Balance
Remaining Balance (generally declines as entries are created in the system)
Rate
Daily/Weekly/Monthly Maximums

The term “Punch Entry” refers to a type of entry representing a time entry by an employee for work that was or is currently being performed. There are different types of punch entries including non-client/service code related (e.g. administration, residential or day program service) and client/service code related (e.g. hourly services).

The term “Attendance Entry” refers to an entry type representing that a client received services by attending a program. Residential, Day and Group Services are examples of programs that provide service to a client when they are in attendance.

The term “EVV” refers to a method used to verify visit activity and service provision for Direct Care/Support, Home Health (Skilled Service), Home Care (Personal Care Service) and Hospice (End-of-life Service). EVV is the means of electronically verifying that an employee/caregiver was physically present with the client.

The term “Profile” refers to a core entity type that may represent a login. Exemplary profile types include:
Company (login not supported)
Funding Source (login not supported)
Employee
Client
Guardian
Case Worker
Day Program
Residential Program
Parenting Program
Group Service

The term “Entry” refers to a core entity type representing a financial transaction. Exemplary Entry types include:
Punch Entry
Attendance Entry
Client Funding Account Entry
Payroll Entry
Billing Entry
Schedule Entry

The term “Account” refers to a core entity type that acts as both a container/ledger for entries and also defines a relationship between two profiles in the system. Common account types include:
Employee Service Account
Client Service Account
Client Funding Account
Master Service Account

The term “Batch” refers to an accumulation of multiple entries (Punch or Attendance Entries) that are processed together.

The term “Rollup” refers to an accumulation of multiple entries that need to be billed together on a single invoice per funding source rules.

The term “Role” refers to a security mechanism that gives an employee profile additional privileges to work with the entities (entries and accounts) for a specific cost center. An employee can only have one role per cost center, but they can have different roles for different cost centers (e.g. Supervisor of one cost center and auditor of another cost center). A role generally allows a user expanded privileges for existing entities in the system, but not the ability to create those entities. The exception to that is Accounts. A role is required to create accounts. Roles may include:
Supervisor
Employer
Auditor
Payroll
Billing
Authorization
View Only

The term “Permission” refers to a security mechanism that gives an employee profile additional privileges to create entities in the system. The difference between a permission and a role is that a permission is not for any one cost center. Permissions may include:
Case Worker Admin
Cost Center Admin
Employee Admin
Holiday Schedule Admin
Residential Program Admin
Day Program Admin
Group Service Admin
Import Admin
Client Admin
Funding Source Admin
Permission Admin
Role Admin
Parenting Program Admin
Training Admin

The term “COA Module” refers to a foundational module of the computing platform 100 that manages other modules and defines relationships as described herein, and specifically may manage:
Profiles
Entries
Accounts
Service Codes
Cost Centers
Batches

The term “Payroll Module” refers to a module that is responsible for the creation and processing of payroll batches. This module processes employee punch entries and creates their associated Payroll entries. This module is responsible for evaluation and determination of overtime (OT) and is the integration point for external payroll systems.

The term “Billing Module” refers to a module that is responsible for the creation and processing of billing batches. This module processes client funding account entries and creates their associated billing entries. This module utilizes service code settings to determine how entries should be billed and it also manages rollups. This module is the integration point for company accounting system and funding source billing system.

The term “Scheduling Module” refers to a module that is responsible for the creation and processing of billing batches. This module processes client funding account entries and creates their associated billing entries. This module utilizes service code settings to determine how entries should be billed and it also manages rollups. This module is the integration point for company accounting system and funding source billing system.

The term “Authorization Module” refers to a feature of the COA Module, or an authorization function, responsible for managing authorizations and client funding accounts. Only users with the Billing or Authorization role (or super users) can access the Authorization module/function.

The term “Settings Module” refers to a module responsible for managing many of the user definable settings and entities. Items under this module include:
Funding Sources/Service Codes/Regions
Holiday Schedules
New Posts
General Activity Options
Training Module
Message Templates
EVV

The term “Reports Module” refers to a module where all of the canned reports live.

The term “Import Module” refers to a module where various imports (bulk load) live.

The term “Messaging Module” refers to a module that controls system and end user generated messages and includes the Notification Engine.

The term “Notifications Engine” refers to a feature of the Messaging Module responsible for sending notifications to end users when messages are generated. May support SMS, Email and Internal Application notifications.

The term “Employee Service Account” refers to an account that may:
Link an employee to another profile (e.g. client or residential/day/parenting/group service program) so that they can make punch entries for it
   Account also defines the service code, cost center and pay rate
Act as the ledger or bucket where employee punch entries and payroll entries will accumulate

The term “Client Service Account” refers to an account that:
Links a client to another profile (e.g. residential/day/parenting/group service program) so that attendance entries can be created for them
   Account also defines the service code and cost center
Acts as the ledger or bucket where client attendance entries will accumulate

The term “Client Funding Account” refers to an account that:
Links a client to a funding source
   Account also defines the service code and cost center
Acts as the ledger or bucket where client attendance or employee service (client portion), authorizations and billing entries

A “Participant” as discussed herein generally refers to clients or people receiving some form of healthcare, mental health, or care-related service. An “Employee” as discussed herein is associated with an Employer/healthcare Provider.

### Detailed Discussion of System Architecture

Returning to FIG. 1A, the computing platform 100 may service an end user portal 115, which may be accessible by a device 112 such as a laptop, desktop computer, smartphone or any mobile device. The end user portal 115 may include a browser-based application and define a user-interface (UI) (not-shown) to interact with various end users. The UI of the end user portal 115 may be embodied as different interfaces depending upon where the end user portal 115 is being accessed and who is navigating the application (e.g. the patient, or healthcare provider employee). In other words, the end user portal 115 may be configured for an Employee, or Employer, or other user. The end user portal 115 may drive time entry tracking and identification of patients, among other features described herein. The computing platform 100 may further include an administrator device 114 which may execute an administrator portal (not shown) to manage aspects of the COA Module 102 and the care modules 104.

The network 122 shown may include any network capable of transmitting communications from one device to another device such as, e.g., the Internet, a virtual private network, a local area network, a wide area network, a Wi-Fi network, a cellular network, or any combination thereof. The network 122 allows the various components of the computing platform 100 to communicate with one another.

In some embodiments, the care management application 116 of the computing platform 100 includes the chart of accounts (COA) Module 102. The COA Module 102 may be configured as a management application that manages accounts and core entities of the computing platform 100 such as profiles, Employees, Healthcare Providers, time entries, batches, service codes, patients, funding sources, and the like, where profiles may define employees, clients, funding sources, guardians, case workers, res programs, Group Services, Parenting Programs, and day programs. Profiles may further be associated with logins and/or users, and batches may define groups of time entries that need to be paid or billed.

The COA Module 102 may function akin to an accounting system, and manage the relationships between the core entities implemented as objects and/or parameters by defining or generating accounts, as described herein. For example, an account of the COA Module 102 may be utilized to link two profiles together; e.g. define a relationship between a patient and a healthcare provider employee. The COA Module 102 may further define one or more service codes associated with the account that define the scope of work the healthcare provider employee is authorized to provide to the patient. The COA Module 102 may further define funding sources for the account such as Medicaid and/or other public or private sources of funding for healthcare bills generated by the healthcare provider employee. For further illustration, FIG. 1B depicts an exemplary entity relationship diagram associated with aspects of the COA Module 102. FIG. 1C depicts possible object entity types associated with the COA Module 102.

Accounts as generated using the COA Module 102 may define containers for time entries/punches for healthcare employees providing care to a patient. A transaction of the COA Module 102 may tie together two entries: a debit, and a credit.

In some embodiments, the COA Module 102 utilizes a single tenant framework/model. To illustrate, a first instance of the COA Module 102, defining one or more accounts, may be generated for a first healthcare provider, and a second instance a COA Module 102, defining one or more accounts may be generated for a second healthcare provider, each of the instances accessible via the care management application 116 using a PaaS and/or SaaS framework. When implemented via SaaS and/or PaaS, the COA Module 102 is configured to be flexible to accommodate different systems and states, while taking into account the important security and privacy concerns associated with healthcare data. Using accounts as described, an unlimited number of relationships may be defined between the different core entities. In some embodiments, the COA Module 102 may be implemented using .Net, such as version 4.5, may include an NBC framework and/or an entity framework. The COA Module 102 may further leverage one or more Microsoft APIs and frameworks.

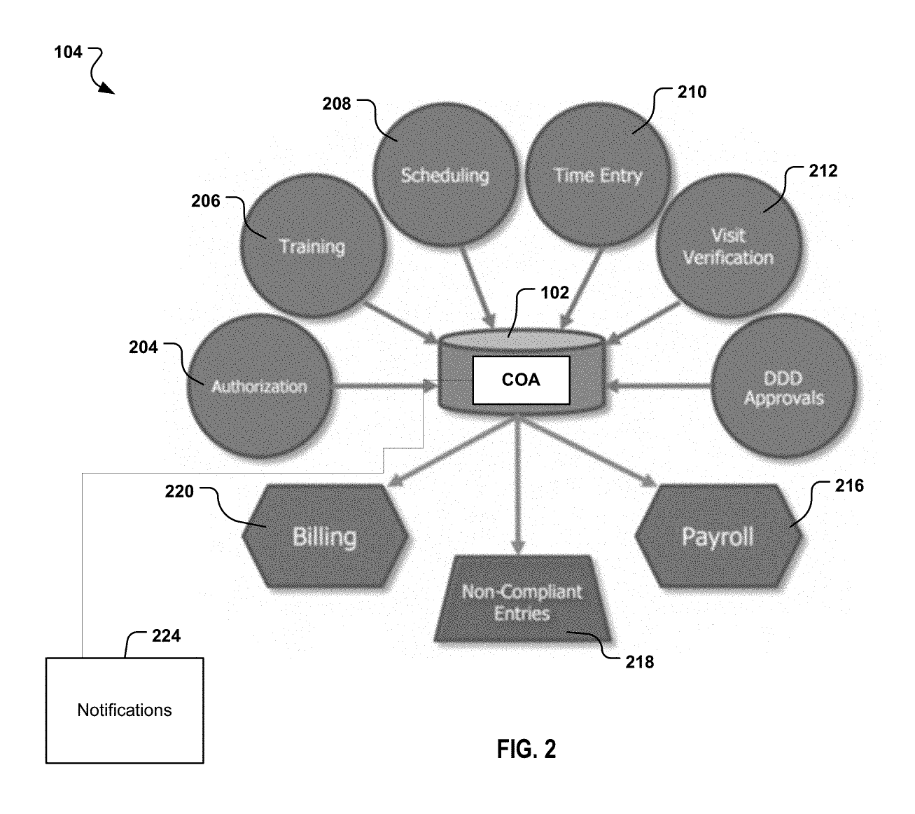

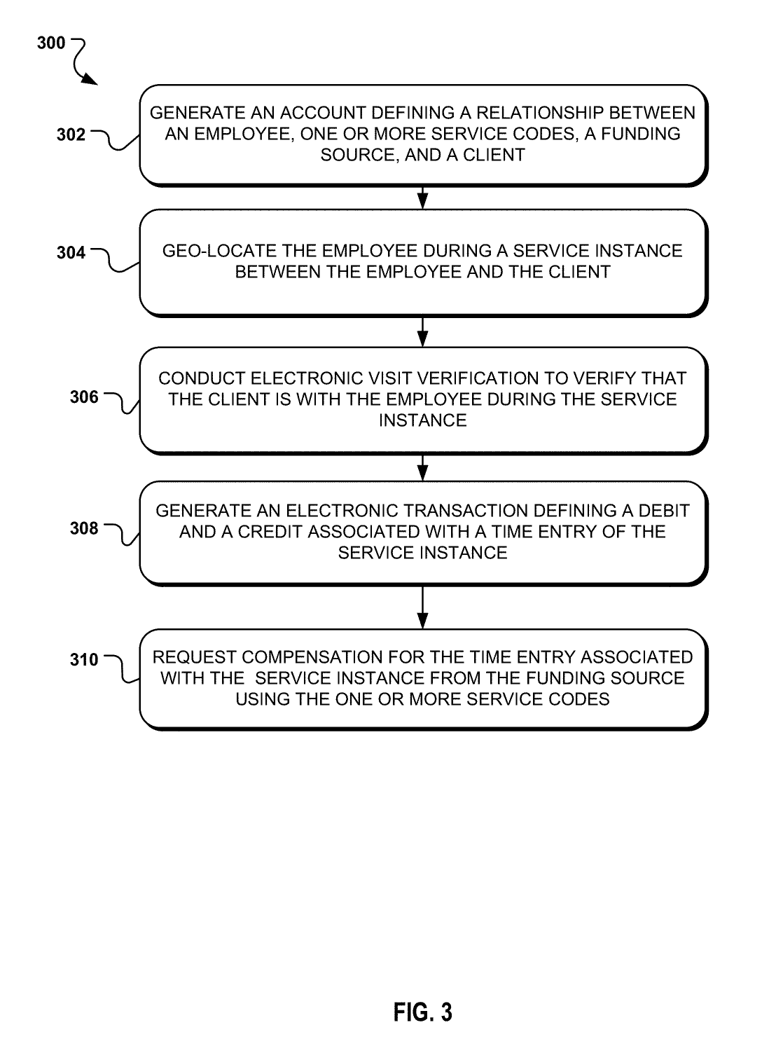

The care management application 116 of the computing platform 100 may further include a plurality of care modules 104 which may be managed by and otherwise interact and/or integrate with the COA Module 102. As shown in FIG. 2, the care modules 104 may include an Authorization Module 204, a Training Module 206, a Scheduling Module 208, a Time Entry Module 210 which may be configured to include time management and attendance, a Visit Verification Module 212, a Payroll Module 216, a Non-Compliant Entries Module 218, a Billing Module 220, and a Notifications Module 224. As illustrated in FIG. 2, the COA Module 102 may be operable to manage data associated with the care modules 104 and otherwise integrate the different care modules 104 together as described herein. One possible process for implementing the care management application 116 including the COA Module 102 and care modules 104 is described in FIG. 3 and discussed below.

The Authorization Module 204 may be configured to serve as a real-time data management and reconciliation system for Medicaid, Medicare, and other service code based insurance authorizations and may be accessible by users and funding sources. The Authorization Module 204 may integrate (via API or custom integration) with, or replace existing, funding source authorization databases, MMIS or other systems of record to import data directly. The Authorization Module 204 provides role-based portals (such as the portal 115) to users including employees and funding sources to effectively manage service authorizations or plans of care. The Authorization Module 204 may be configured for: providing a system of record for healthcare providers, employees, and funding sources to access and maintain service authorizations; tracking real-time declining balances of authorizations so the funding source and patients or employees are aware at all times of the amount of approved service that remains on the authorization; facilitating creation of new service authorizations; deactivating old service authorizations; editing current service authorizations; auditing and reporting on service authorizations; increasing or decreasing service units or dollars on current authorizations in real-time; and ensuring compliance of claims billed.

The Authorization Module 204 may be loaded with or otherwise access account budgets, authorizations, or plans of care from a funding source system 120. The Authorization Module 204 may also integrate with existing funding source 120 databases or other systems. Using the Authorization Module 204, it may be verified whether a user such as a patient has a current authorization to receive certain care from a healthcare employee.

Additionally, the Authorization Module 204 may be operable to verify both the type and amount of services to be provided prior to authorizing payment for goods or services on behalf of a healthcare provider. Documentation in a funding source system may be required for authorization in order to calculate reimbursement to healthcare employees for prior authorized services. Using the computing platform 100 and the Authorization Module 204, service events may be entered into a system designated by the funding source prior to billing in order to receive reimbursement. An employee, or company, or other user which may be associated with healthcare provider system 118 may be given access to a secure portal where they can manage and view information based on the role security and business rules defined by a funding source. The Authorization Module 204 allows a healthcare provider to view the following in real time: service codes authorized for services; original authorized unit or dollar amount; real-time authorization units or dollars remaining balance; daily, weekly, monthly, quarterly, or annual authorizations limits set by the program; original start date of authorization; expiration date of authorization, and a bill rate for authorization.

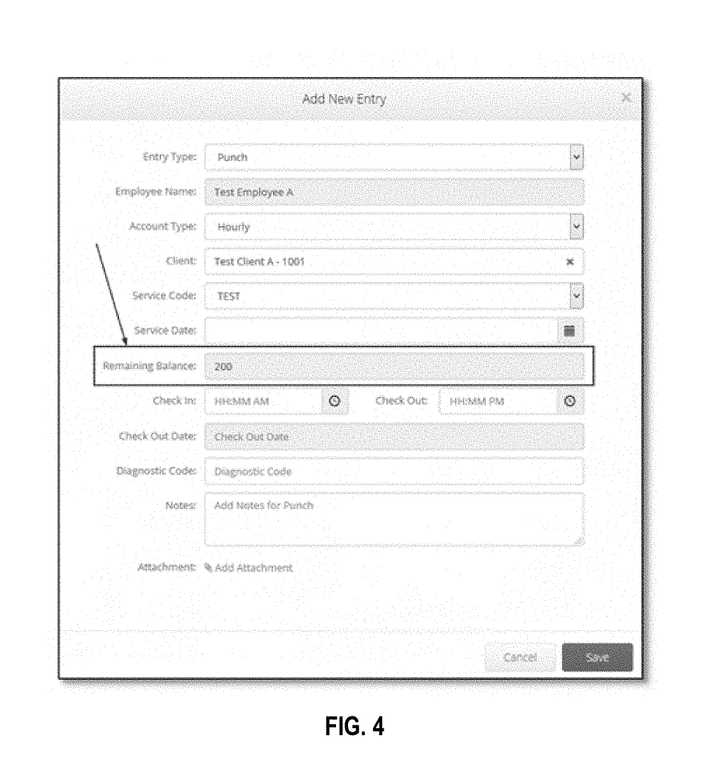

A company or other end user can be given view-only access of their respective authorization based on business rules defined by the program. For example, employees associated with an employer may generate time entries using an employee portal. As time entries are made, employees may view a remaining real-time authorization balance every time they generate a time entry, as shown in the screenshot of FIG. 4.

After, and in some embodiments only after valid authorization, employee time entry, and electronic visit verification has been received, the care management application 102 may generate billing to the funding source system 120. This may improve compliance for a service or good that is billed. In some embodiments, adjustments can be made using the Authorization Module 204 including voiding prior authorizations and data collection, as necessary. In this capacity, the Authorization Module 204 improves the likelihood that claims submitted for payments for goods and services are authorized in the current budget/expenditure plan associated with an employer or healthcare provider system 118.

The Training Module 206 may be configured to improve training compliance for employees (which may be associated with a healthcare provider) and to proactively alert regarding training deficiencies before expiration of e.g. certifications. In particular, the Training Module 206 provides configurable options for designing training profiles, including specific service lines/service codes, employee types, or individuals in service. The Training Module 206 is operable for monitoring and reporting including: monitoring renewal and expiration dates for training certifications; generating notifications for various users; integrating with other care modules 104 to provide that e.g. employees are scheduled for services in which they are trained and authorized; and reducing the probability of non-compliant time entries. The Training Module 206 further provides access to live calendar applications to schedule training and other resources.

In some embodiments, service code training profiles can be created with the Training Module 206 for service codes, with such profiles configured to meet contractual requirements associated with individual service codes. For example, various healthcare providers assigned with a service code “HAB” could have a set of required trainings in their training profile that their Employees must complete. Once these Employees satisfy their respective training requirement(s), they may be eligible in the system to perform work. One example of an HAB service code training profile could include the following service codes: Medication Training; Background Check; and/or CPR/First Aid Training.

To further illustrate, a hypothetical healthcare provider's training profile may define authorized service codes, abbreviated as “HAB”, “RSP”, and “SL”. Specifically, the HAB service code training profile may include: Medication Training; Background Check, and CPR/First Aid; the RSP service code training profile may include CPR/First Aid Training; and the SL service code training profile may include: lifting and Transferring Training, and CPR/First Aid Training. In other words, the Training Module 206 may be configured to define what training is associated with individual service codes.

In one embodiment, using the Training Module 206, in order for an Employee of a healthcare provider to provide services to a patient on behalf of a healthcare provider, they may be required to have completed the trainings that correspond to a particular service code profile. This assists the healthcare providers to utilize Employees that qualify for all services to be performed, as well as Employees that may only qualify for select services. For example, the hypothetical healthcare provider that has the training profile described above may have only one Employee that the hypothetical healthcare provider employs to provide services under the HAB service code, but could have three (3) additional Employees that are qualified to provide RSP and SL (and not services under the HAB service code). This functionality enables healthcare providers to maximize staffing opportunities, an essential component of self-direction and direct care, due to significant staff shortage potential.

The Training Module 206 may further be configured to define trainings in an Employee training profile that may not be defined in the healthcare provider profile. This function may be useful for one-off trainings that the Health Care Provider may want to assign to Employees. Assigned trainings may or may not be relevant to an individual Participant's needs. For example, the Health Care Provider can use this functionality to assign a one-time training to Employees on Department of Labor (DOL) Regulations such as FLSA and HIPAA.

The Training Module 206 may be operable to monitor renewal and expiration dates for training certifications and notifies Employees or other designated personnel that training certificates are approaching expiration or have already expired. Notification alerts can be configured to notify designated compliance personnel at intervals designated by employer, or the Participant. Notifications can also be configured to repeatedly alert Employees at designated intervals. Furthermore, when integrated with the Scheduling Module 208, the Training Module 206 can disallow any Employee to be scheduled to work with any Participant for whom they do not match the Participant's training profile.

In some embodiments, when integrated with the Time Entry Module 210, the Training Module 206 can disallow an Employee to make time entries if they do not have the required trainings needed to provide service to the Participant. For these instances, the computing platform 100 can be configured to provide notices to the Employee, other designated personnel. All notifications and alerts can be transmitted via an employee portal, the employer portal, an administrator portal, e-mail, or text message.

In addition to helping facilitate compliant service, the Training Module 206 may integrate with a learning management system (LMS). This system hosts online trainings and testing, creates certificates, and monitors training compliance. For example, training videos and corresponding tests approved by the funding source can be loaded into the LMS. Employees can view these trainings and take training-related tests through an interactive video player. Subsequently, the Training Module 206 can score tests and create certificates for Employees that pass. These certificates may be imported automatically into the Training Module 206 database for record-keeping and compliance tracking. Employees can also print certificates from the system if they would like to possess physical copies. Lastly, the Training Module 206 can integrate with external training and learning management systems, and online trainings and curricula can be delivered as requested.

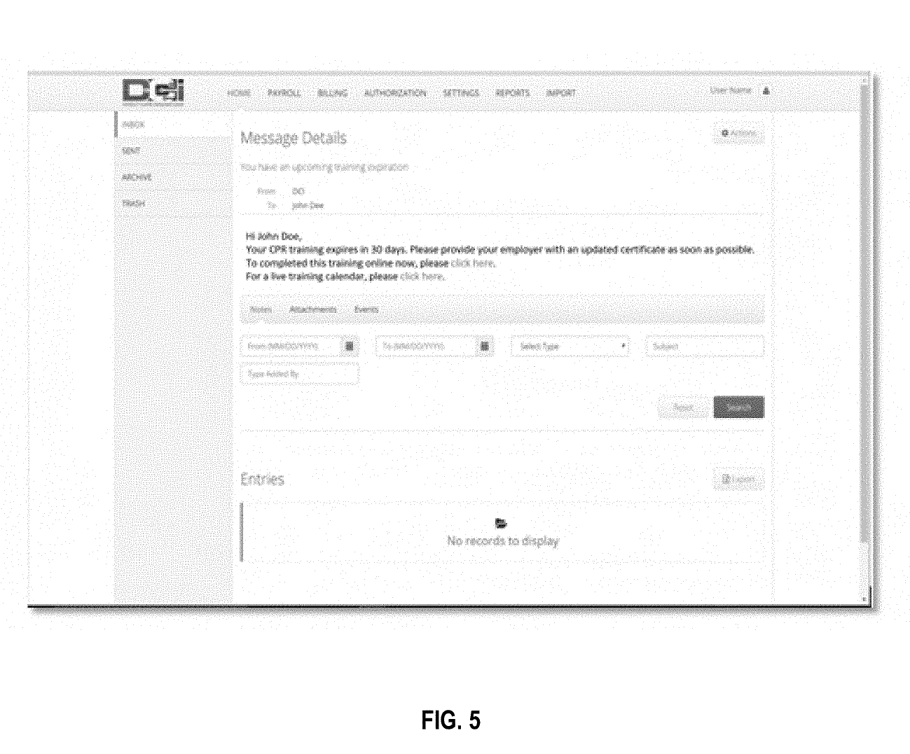

In some embodiments, when the Training Module 206 is integrated with the Time Entry Module 210, the computing platform 100 can send training alerts to the Employee each time they login. This notification can help Employees ensure they remain in training compliance. A ‘training due’ notification prompts the user to click the link to renew their expiring training. If online training is not available for expiring trainings, the Training Module 206 can be configured to provide the employee a link to a live training calendar for them to schedule an in-person class for their training renewal. A screenshot of a training notification is shown in FIG. 5.

The Scheduling Module 208 may be configured to integrate with the Authorization Module 204 to create schedules that maximize services to Participants without exceeding authorization. In some embodiments, the Scheduling Module 208 is a value-added service that employers can choose to use or not use depending on their preference. The Scheduling Module 208 enables employers to effectively manage Employee hours, overtime, Employee rotations, and shift trading. The Scheduling Module 208 enables Employees to create client schedules that maximize services to each client. The Scheduling Module 208 also accommodates online shift trading between Employees. In some embodiments, the Scheduling Module 208 uses two schedule paradigms (Future Schedules and Active Schedules) for schedule creation and schedule management.

In some embodiments, the Scheduling Module 208 enables future schedule creation such that an Employer creates a schedule using a weekly calendar based on the services they would like to provide. An Employer can create multiple weekly schedules if they would prefer to schedule services further in advance (e.g. one month at a time). The following points highlight some of the possible features of future schedule creation through the Scheduling Module 208: the future schedule may be instantly compared to the Client's authorization to ensure that the services being scheduled are within their authorized plan of care; if the schedule exceeds the authorized plan of care, the Employer may not be allowed to complete the schedule. The Employer may then be notified that the schedule they are attempting to create exceeds the authorized plan of care. The Scheduling Module 208 can enforce hourly, daily, weekly, and monthly limits on schedules. These rules can be configured and enforced differently for each individual service code.

In some embodiments, if the schedule is within authorization limits, the Employer can proceed to choose staff to fill each scheduled hour of service. The Employer may also choose Employees to associate with each scheduled shift until staff hours are accurately matched to desired service hours. Employer may receive a warning if they are scheduling staff into overtime. In some embodiments, when integrated with the Training Module 206, if the Employee's training profile does not satisfy the service codes required training, scheduling may be disallowed. If an Employee is not current on all certifications, they may not be scheduled and the Employer may be notified about their deficiency. For example, they would receive a notification “Jane Doe cannot be scheduled, CPR certification has expired.” In some embodiment, once an Employee has been scheduled, they can be notified via the time entry portal, the participant portal, e-mail, or text message.

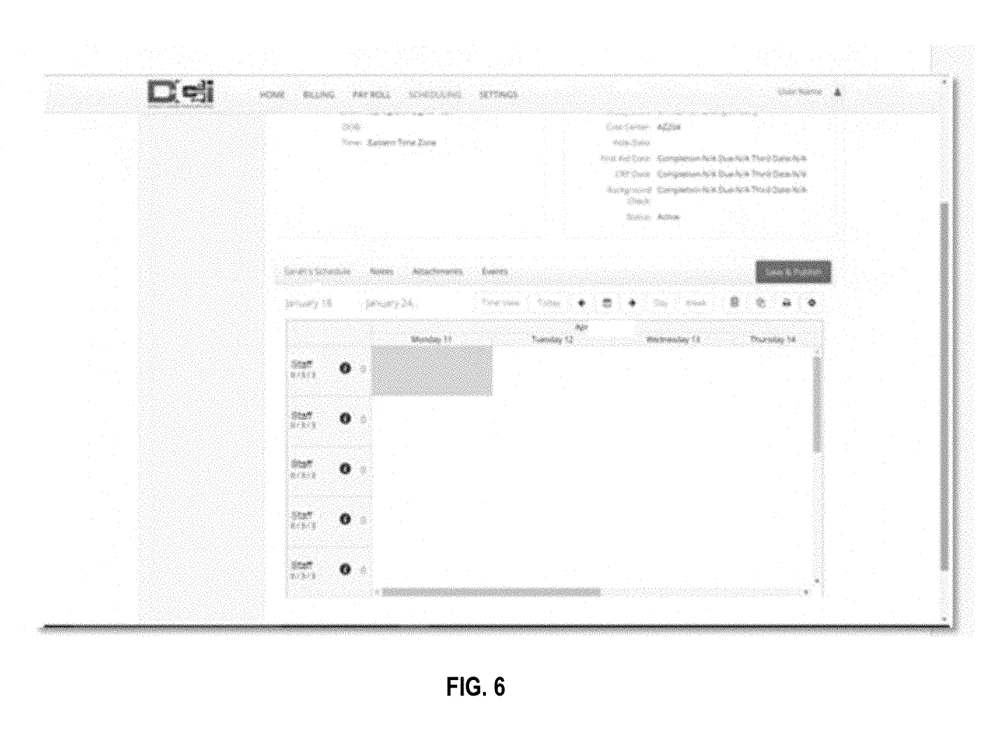

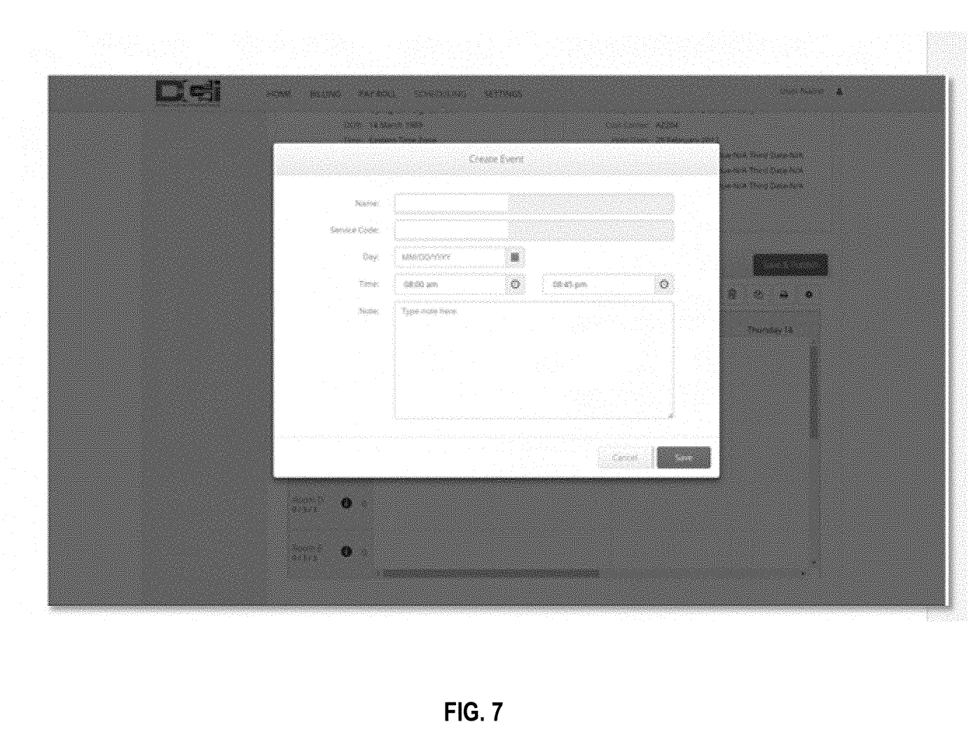

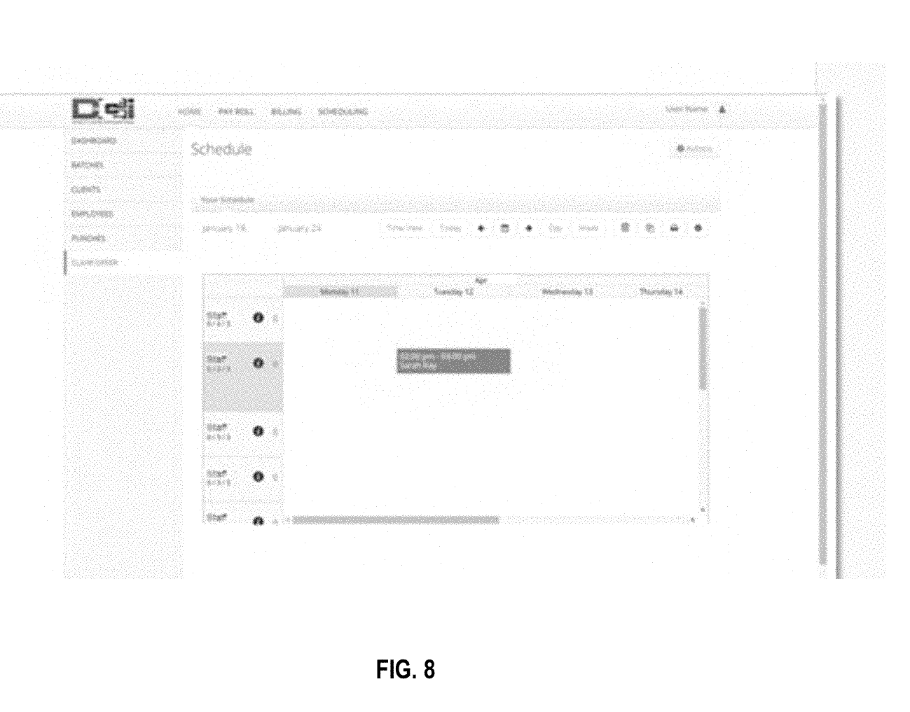

Screen shots depicted in FIGS. 6-8 illustrate user friendly scheduling when implementing the Scheduling Module 208. In FIG. 6, an Employer selects from a calendar as shown. In FIG. 7, the Employer may then schedule a selected Employee. In FIG. 8, once the Employer saves the scheduling of the Employee, the Employee is added to the schedule as shown.

In some embodiments, Employers may have the option to reuse a previous schedule. This function allows Employers who have consistent staffing schedules to save time by re-creating the same schedule each week, or month.

In some embodiments, the Scheduling Module 208 includes an active schedule manager to monitor Employee actual time versus scheduled time. In particular, the active schedule manager may be integrated with the Time Entry Module 210 to match actual time entries to desired scheduled times. Once a future time schedule has been approved and the date and time of the first scheduled shift arrives, the schedule becomes active in the Active Schedule Manager. The Active Schedule Manager may show the desired schedule and whether or not Employees are checked-in according to the schedule in real-time. The Active Schedule Manager can be configured to notify Employees, if there is any scheduling variance. Notifications are transmitted via the notification engine.

In some embodiments, the Scheduling Module 208 can be configured to require Employer acceptance of a traded shift before it can be updated. It can also be configured to reject any shift claim that would put an Employee into overtime. If an offered shift is not claimed, it may revert back to the original Employee that was scheduled within an assigned number of hours prior to the shift. For example, if John Doe offers to give up a shift, but no other Employee claims it within twenty-four (24) hours of the scheduled shift, the shift reverts back to John Doe. A notification is then sent to John Doe as well as the Employer. The timeline for shifts reverting is configurable.

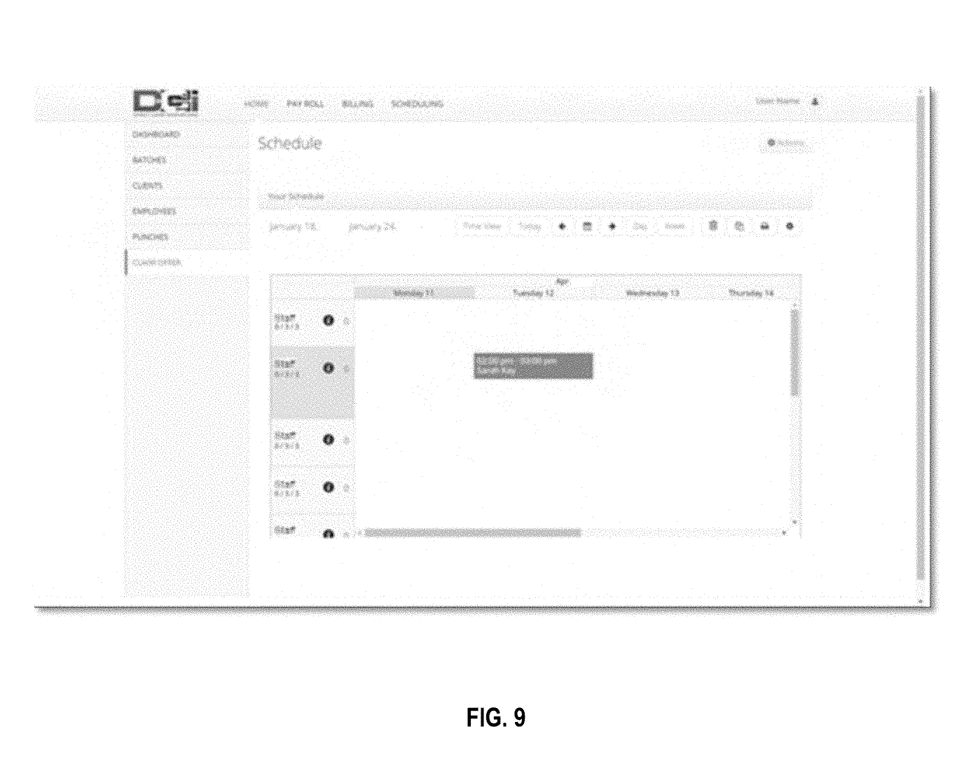

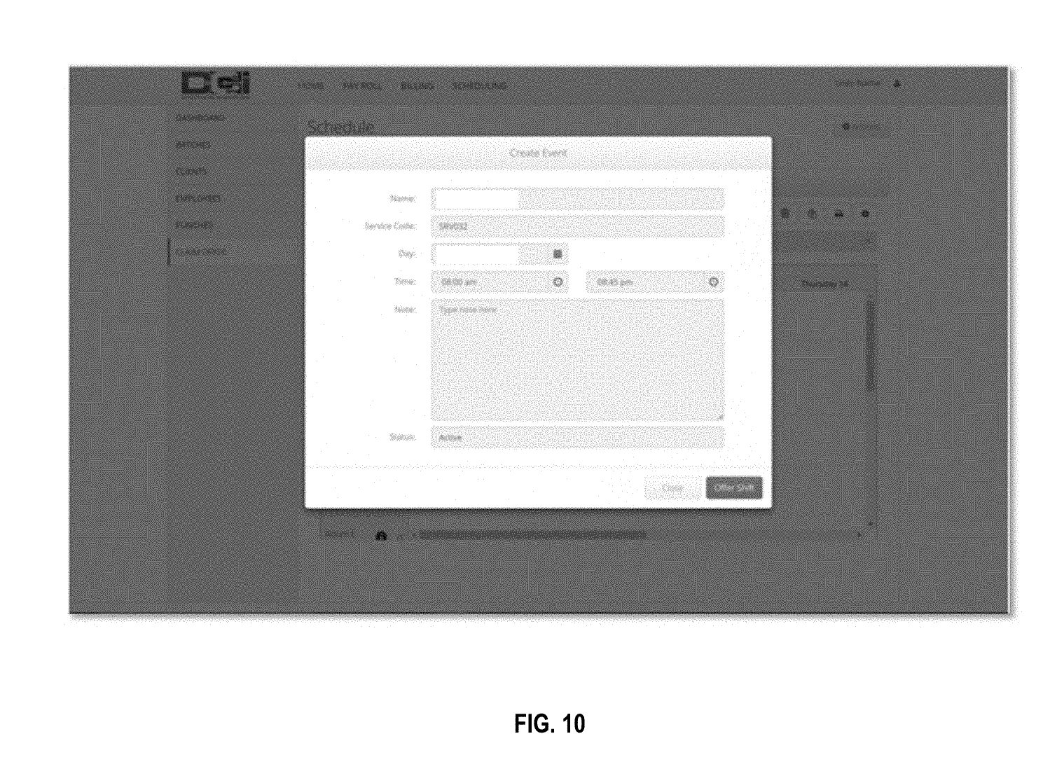

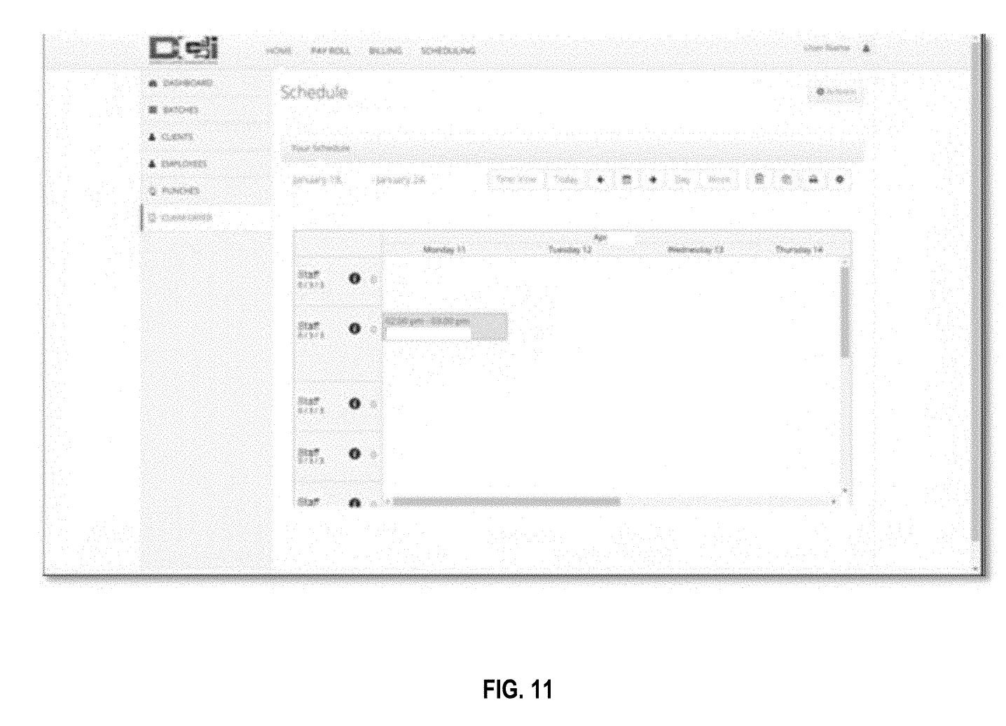

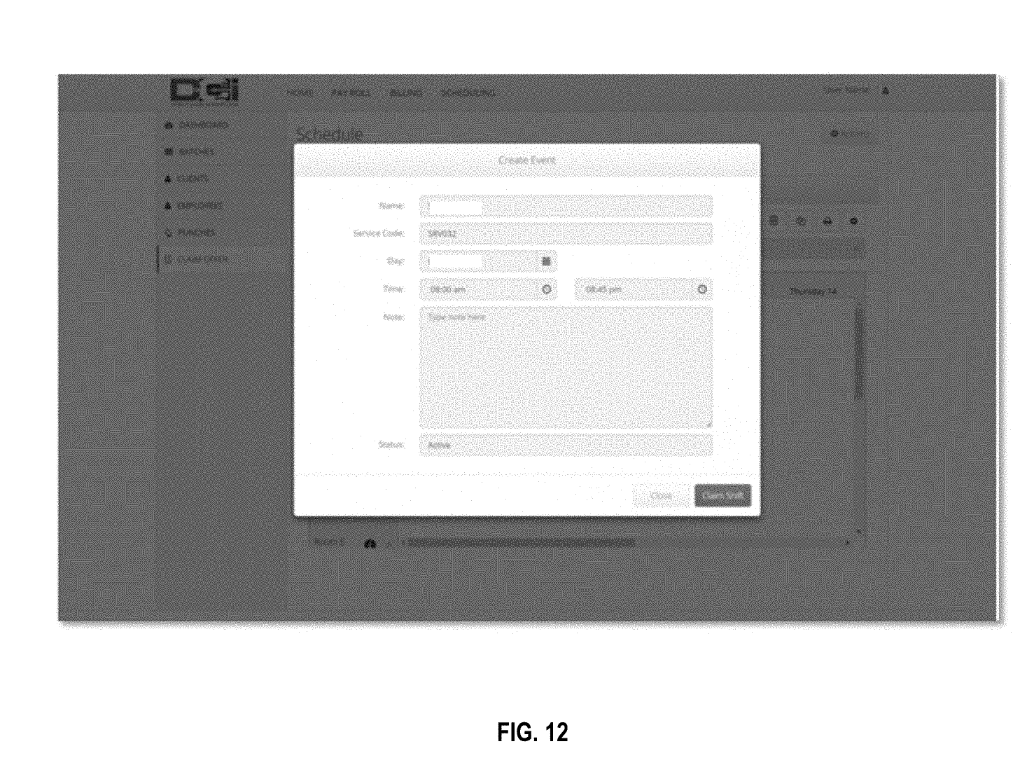

Screenshots of the employee portal for trading shift process are shown in FIGS. 9-12. In some embodiments, referring to FIG. 9, the Employee clicks on a scheduled shift they would like to trade. Referring to FIG. 10, the employee may then be asked if they would like to offer up the shift for other Employees to accept. Referring to FIG. 11, once the Employee offers up the shift, it may appear as available on a calendar defining open shifts. Other employees linked to the Participant can claim these open shifts by clicking on the shift highlighted in yellow in the screenshot of FIG. 11. The Employee may then be presented with a confirmation to claim the offered shift as shown in FIG. 12. Once an Employee claims the shift, the shift is either given to the Employee automatically, or a request to approve the claimed shift may be sent to the Employer.

Utilizing the Time Entry Module 210 and the Visit Verification Module 212, any number of technical options may be implemented to enable time entry recording and visit verification (verifying that an employee is actually providing a care service to a patient) for Participants and their employees in different situations. For example, referring back to FIG. 1, a mobile application (Mobile App) 106 may be implemented and configured for tracking time and visit verification for services delivered remotely in homes or community settings via e.g. a smartphone. In some embodiments, the Mobile App 106 provides a double verification process that requires both Employee and Participant verification to ensure services are being provided appropriately, thereby reducing fraud and waste. The double verification process begins with the Employee clocking in on the Mobile App 106 using a real-time running shift clock. The Participant must then verify that they are physically with the Employee within two (2) minutes (this time is configurable and can be adjusted to any time limit requested by the funding source). Participant Verification can be performed via any of the following methods: entering a unique Participant-chosen PIN; an Employee taking a photograph of the Participant, which is time-stamped; and/or with an E-signature taken on the mobile device. All verification methods may be configurable and hierarchical. Desired configuration and hierarchy may be determined by funding source during implementation.

In some embodiments, the Mobile App 106 may be configured to prompt for additional verifications as often as the funding source desires. For example, if the funding source would like the Employee to re-verify that they are physically with the Participant every forty-five (45) minutes the Mobile App 106 can be configured to perform this request. Under this example, the Mobile App 106 would notify the Employee to have the Participant enter their verification every forty-five (45) minutes. All verification options are configurable and up to the discretion of the funding source.

In some embodiments, the Mobile App 106 may be configured with enhanced tracking capabilities. For example, the App may track physical location through geo-location functionality, a critical component of an electronic visit verification (EVV) process.

In some embodiments, the Mobile App 106 provides functionality for tracking transportation time and mileage incurred within a shift by an Employee. Specifically, within the Mobile App 106, the Employee can select a function called “Client Transport”. This function initiates the tracking of total trip time/duration and mileage traveled during a shift. This functionality is enabled with geo-location tracking via e.g. Google Maps within the Mobile App 106. When the Employee completes their transportation, they can then click to end “Client Transport” tracking, and continue with their shift. If the Employee has reached the end of their shift, they can simply log out of the Mobile App 106. Logging out will end the Employee's shift in the system, and will also end Client Transport tracking that was in progress. For the avoidance of doubt, if an Employee chooses to end their shift, and subsequently decides to log back into the Mobile App 106 to use the tracking functionality, they'll need to re-verify that they are still physically with the Client.

In some embodiments, the Mobile App 106 can also enable tracking of Employee drive time between Participant shifts when traveling from one Participant/shift to another. This function in the Mobile App 106 may be called “Drive”. “Drive” is useful for any programs that pay for time driven between shifts. (This is a configurable feature and does not need to be used in programs that do not pay for transportation between participants.). The Mobile App 106 may further include a real-time running clock, tracking the duration of each Employee's shift. The Mobile App 106 may further include a verification feature such that when an Employee's shift ends, the Employee must re-verify that they are physically with the Participant before they can clock out. The Mobile App 106 may further include real time declining balances. Specifically, the Mobile App 106 may provide access to the current authorization/plan of care balances to both the Employee and the Participant in real-time. In some embodiments, the Mobile App 106 provides configurable settings that allow agencies to apply flexible business rules. The Mobile App 106 may further include a notification engine that allows the Employer to configure notifications on expiring authorization balances, overtime alerts, and other tracking functions within the system. Notifications are flexible and can be configured to meet specific agency needs and workflows.

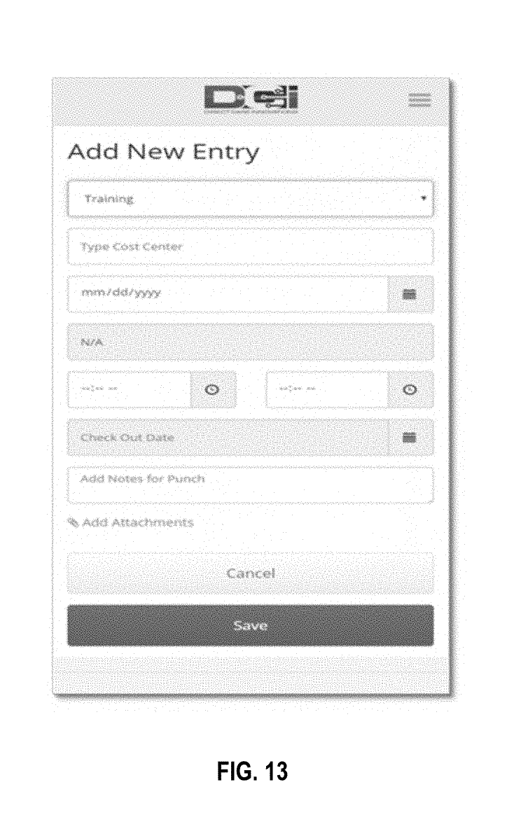

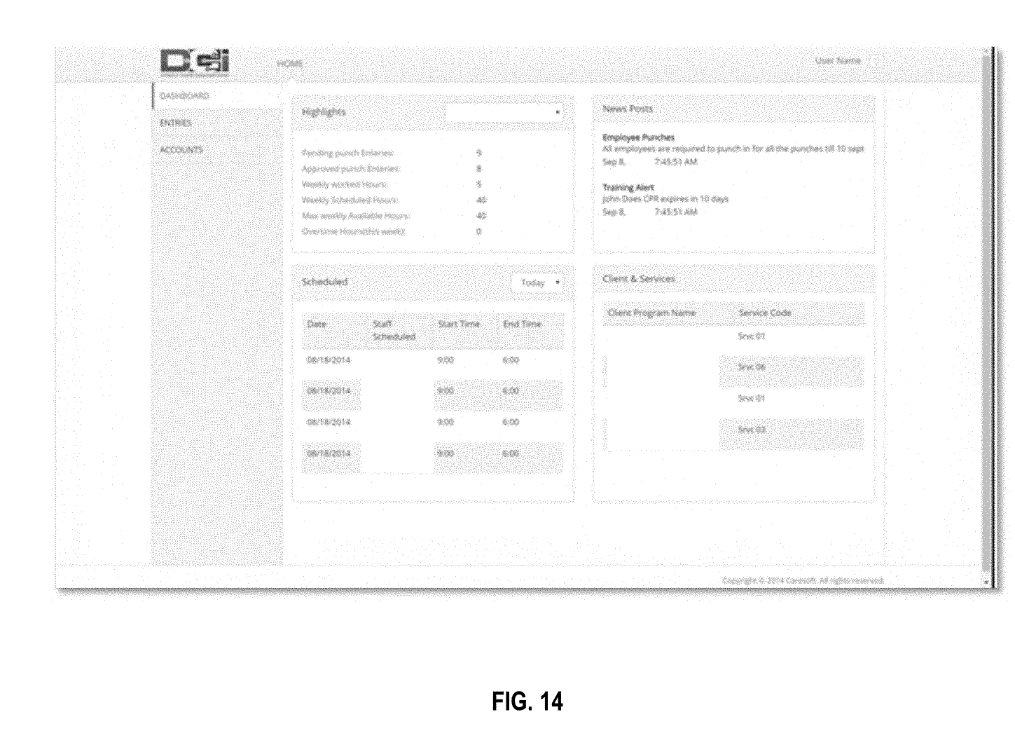

In some embodiments, the Mobile App 106 may include user verification which can be performed via user name and password, quick pin, telephonic, or biometric verification. Referring to the screens shot of FIGS. 13-14, an employee portal interface of the Mobile App 106 may further provide configurable interfaces and dashboards for Employees to track hours, overtime, and services provided.

In some embodiments, the Mobile App 106 may include a double verification process as described herein that may be community based. The Time Entry Module 210 may be integrated with the Mobile App 106 or can be used stand alone for participant verification. Using Employee time entries, the Time Entry Module 210 generates punches for the Participant to verify for approval. Participant verification may be performed through a Client portal. The Client portal is a secure portal that can be accessed with a user name and password or PIN number. The Time Entry Module 210 may verify all time entries against the authorization, the approved schedule (if desired), and the defined program rules before presenting it to the Employer for final approval. This process creates a simple employee time and budget management experience for the Employer. This process also ensures the funding source is never billed for any time entry that is not both authorized and compliant, per the established funding source rules. Business rules can be configured based on waiver types and funding source standards. Configurable time entry business rules can include, but are not limited to:
Entries that require Participant verification;
Entries that require Participant, guardian, or third party verification;
Entries can be limited to entries made on-site, from a designated IP address, or enabled for acceptance from any internet connection;
Entries can also be tracked via Global Positioning System (GPS) capabilities;
Entries can be configured to be accepted throughout a pay period;
Entries can be limited to real-time clock in and clock out;
Entries can be tied to pre-set schedules when integrated with the Scheduling Module 208;
Entries can be limited by authorization availability when integrated with the Authorization Module 204;
Entries can be tied to Employee-Participant ratios when integrated with the Scheduling Module 204;
Entries can be limited by training requirements when integrated with the Training Module 206;
Entries can be approved, denied, or kept in pending status contingent upon configured business rules reconciliations.

The Time Entry Module 210 may provide the following EVV options:
Telephonic integration
   For example, an Employee calls from a phone located at the Participant's home to clock-in and clock-out, thereby verifying the visit took place.
Static IP visit confirmation;
   For example, a Participant initially registers a web-connected device that may include a static IP address. A device such as a desktop computer or tablet would satisfy the static IP address condition. Employees would then be required to clock-in and clock-out from the designated device with the static IP address. The computing platform 100 will then seamlessly verify the visit against the IP address previously provided by the Participant.
Bio-metric device integration;
   An Employee uses a bio-metric device at the participant's home to clock in and out verifying the visit.
Fob integration
   The fob is an electronic keychain device used to confirm that the Employee was physically located with a Participant at times punched into the portal.
Participant Portal Visit sign-off.
   Participants have their own user portal. Any time entries made by Employees are recorded there. Participants can approve or reject punches in the participant portal to ensure visit verification.

The Time entry module 210 provides configurable settings that may allow the Employer to apply flexible program rules. The Time Entry Module 201 includes a notification engine that allows the Employer to configure notifications on expiring authorization balances, overtime alerts, and other tracking functions within the system. Notifications are flexible and can be configured to meet specific Employer needs and work flows. All EVV options are optional and can be configured (e.g. turned on, turned off, or adjusted) based on the requirements of funding source.

In some embodiments, Employees can only enter time for Participants they have been associated with via the COA Module 102. Additionally, Employees can only enter time for pre-populated service codes that the Participant has been authorized for. Multiple pay rates can be set for Employees based on the service code being delivered. For example, Employee Jon Smith can be paid $10 an hour for working with Participant Jane Doe when providing the ‘HAB’ service code, while being paid $9 an hour for providing the ‘RSP’ service code to Jane Doe. This functionality is critical for Employer cost management, as service codes typically contain variable billing rates. This functionality also allows Employers to pay variable pay rates for the same service codes for different Employees. For example, Employee Jon Smith could be paid $10 an hour for working the service code ‘HAB’ with participant Jane Doe, but Employee Bill Smith could be paid $11 an hour for working service code ‘HAB’ with participant Jane Doe.

The Time Entry Module 210 includes an employer portal where Employers can view and approve pending time entries made by their Employees. The employer portal may contain Employee time entry details, including: Employee name, start time, end time, service code punched, and how many units or dollars they used. The employer portal allows the Employer to then approve or reject these entries using a simple user friendly two button option. The Employer either clicks the “A” button for approval or the “R” button for reject. The approval and rejection buttons are color coded for further ease of use for Employers. The time entry module 210 also includes multiple filters and search functions (e.g. presorting of information) that improve the user experience for each Employer.

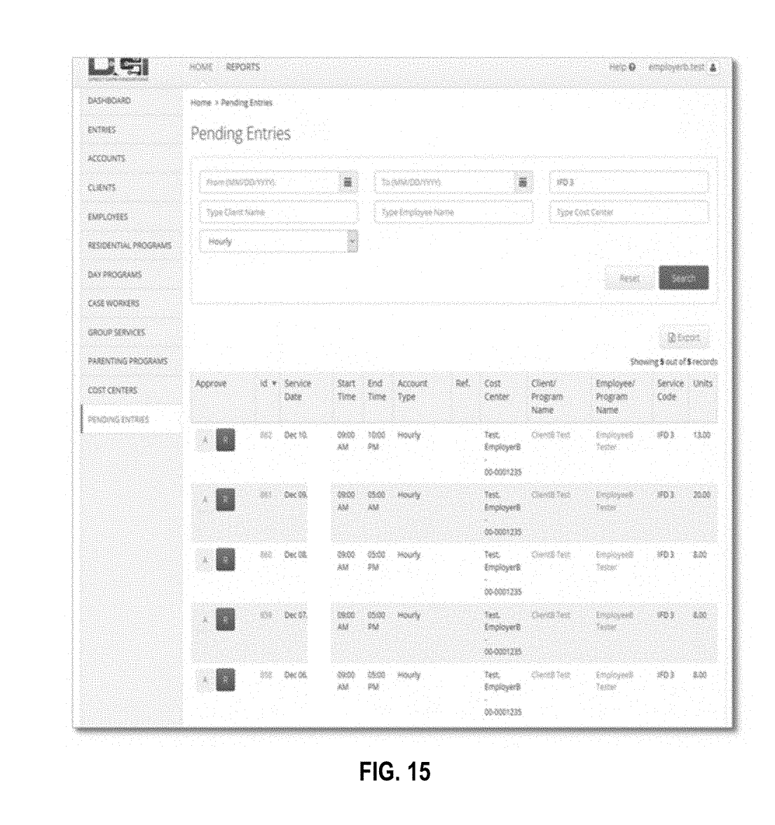

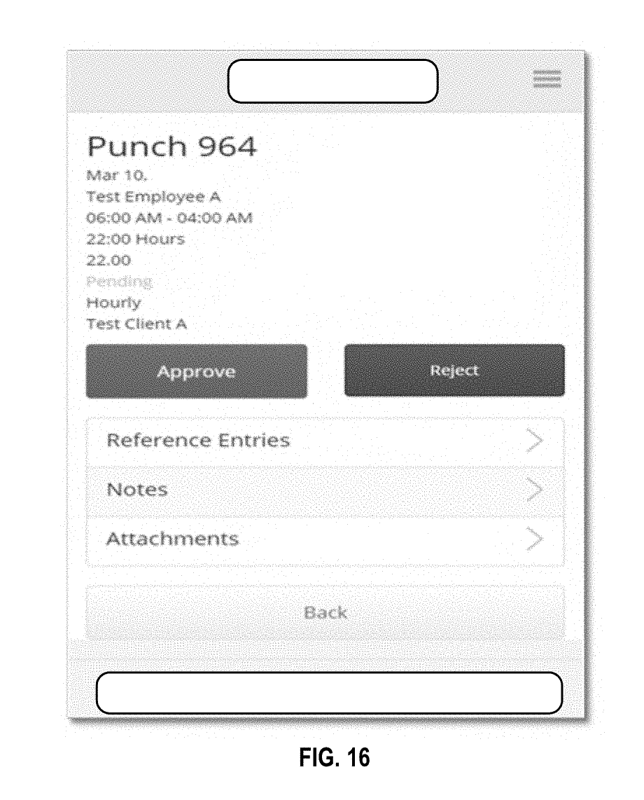

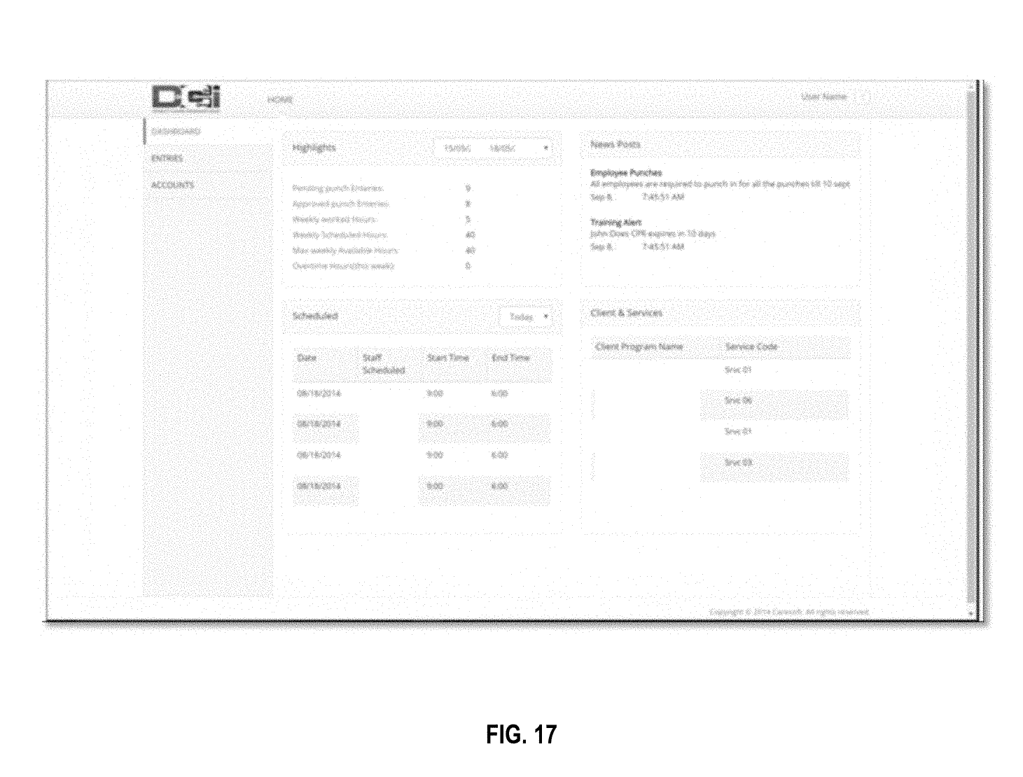

FIGS. 15-17 illustrate the functionality of the Time Entry Module 210 described in the previous paragraph. In FIG. 15, pending entries are displayed. As discussed above, the portal 115 which may be configured for an Employee can be accessed through any internet connected device, and is mobile-enabled. The mobile entry interface is shown in FIG. 16. The employer portal of the Time Entry Module 210 allows Employers to view all Employees and Participant information within integrated Care Modules 104 based on assigned role based security access. Referring to FIG. 17, the Participant portal provides configurable dashboards that enable Participants to easily track key performance indicators (“KPI”).

Referring back to FIG. 1A, in some embodiments the client or end user device 112 may comprise a fob, or key fob, or hardware token and may be implemented to track Employee visits and interaction with patients. In the case where the end user device 112 includes a fob, in which the fob may comprise a programmable hardware device that may be used to confirm that the Employee was physically located with a Participant at times designated on time cards. The fob device may be powered by a watch battery, may generally be of the size of a key chain, and can be carried easily by a Participant or secured in a designated clock in or clock out location.

The fob device generates a unique identification number associated with each minute of every day. For example, at 10:58 am the fob device could display “278 091”, and will change to a new randomly generated number at 10:59 am, such as “156 992”. All numbers generated on the fob are based on a proprietary algorithm. These generated numbers may be tracked by the computing platform 100, for verification purposes. When an Employee is ready to begin a shift, the Employee simply pushes the red power-on button on the fob, which will then display a 6-digit number or other identifier. The Employee writes the number along with the time and service code for the service rendered on a time card. The process may then be repeated upon completion of the Employee's visit, or upon clocking-out. The Employee may replicate this easy process for each Participant visit.

Data associated with the time card including the identifiers may be inputted into the computing platform 100 so that time, service codes, and fob verifications can all be validated prior to processing and billing.

The Billing Module 220 utilizes data inputs from the Authorization Module 204, and the Service Code settings 206 to create billing files and accounts-payable invoices. Specifically, the Billing Module 220 uses time entry data to create billing files. The COA Module 102 ensures:
No service can be billed for the wrong service;
No service can be billed for amounts that exceed available authorized units or dollars;
No service can be billed for the wrong Participant;
No service can exceed hourly, daily, monthly, or annual limits.
No service can be billed for Employees that are not 100% training compliant;
No service can be billed when two (2) Employees attempt to enter service for the same time for the same Participant(s), unless authorized;
No service can be billed without approval and/or visit verification;
No service requiring Participant verification can be billed without confirmed Participant verification.

In some embodiments, the computing platform 100 may further include a Reporting Module (not shown). The Reporting Module provides reporting of all data within the computing platform 100. A variety of standard reports may be generated through the Reporting Module. Administrator and auditor viewing access may be provided to all transactions in all modules using the Reporting Module. Permissions can be assigned for view-only access to funding source service coordinators, auditors, or other external parties as deemed necessary by the Employer.

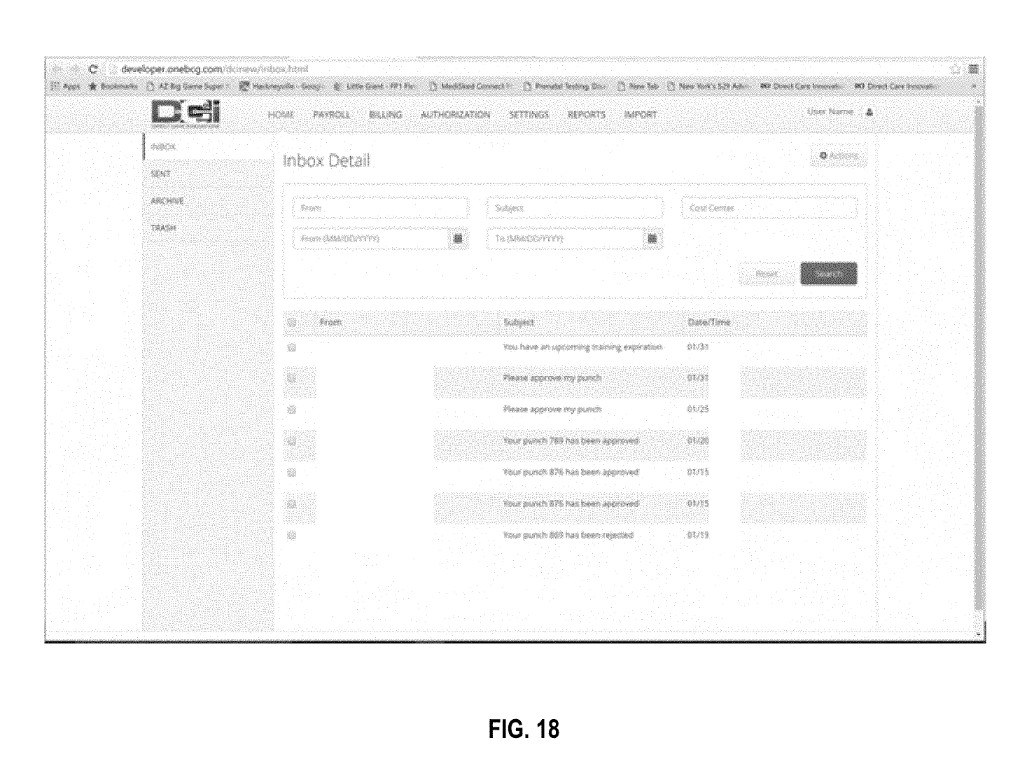

The Notifications Module 224 may include a powerful notification engine that facilitates notification and alerts to Participants, Employees. Notifications can be generated by text message, e-mail, and sent directly to the employer, employee, and administrative portals of the computing platform 100. Notifications can be initiated for events such as: expiring training, over scheduled hours, low authorization balance, or any other data stored within the system. The Notification Module 224 is configurable and highly flexible for all user perspectives (e.g. Participant, Employee,). The level of customization allows the Notification Module 224 to fulfill specific waiver program and stakeholder needs. Referring to FIG. 18, a screenshot of how notifications may appear to a user using the internal messaging system is provided. The user's ‘inbox’ aggregates notifications, alerts, data updates, and required actions from modules across the computing platform 100 in one place.

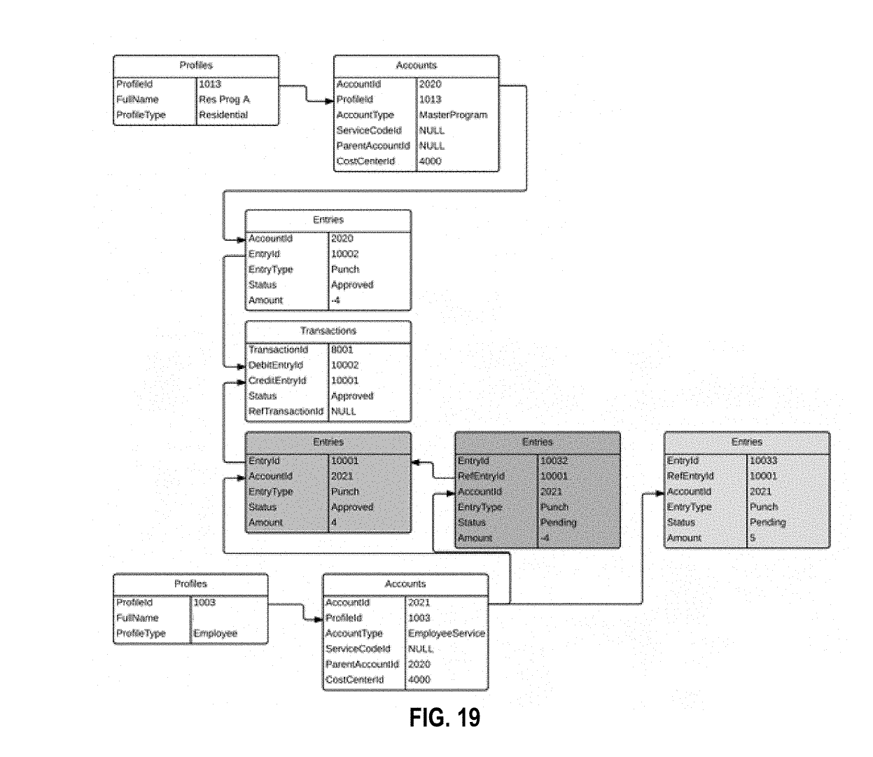

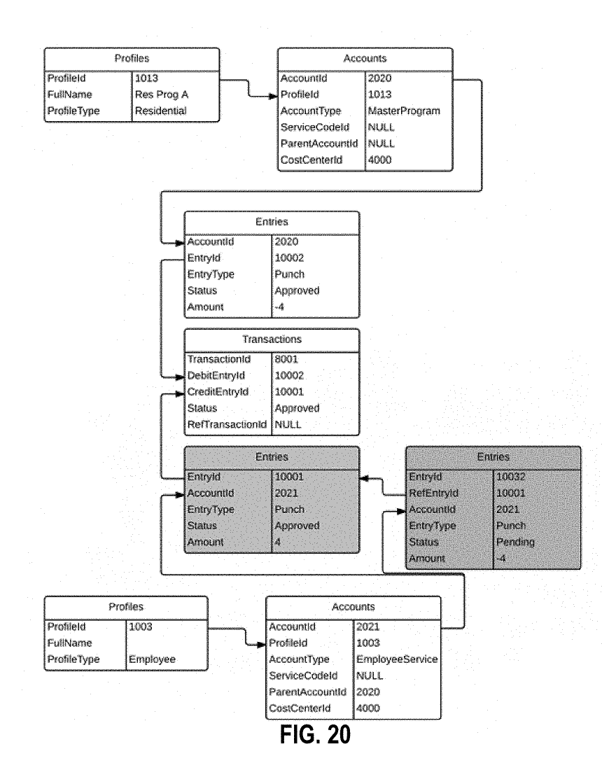

Referring to FIGS. 19-20, payroll processing using the Payroll Module 216 will now be described. The COA Module 102 may define a “compensating entry,” which may involve correction of an approved entry by replacing the original entry with a new entry. In particular, because the original entry may have been approved and effected balances in other accounts, the original entry may be backed out with a reversing, or compensating entry. In short: compensating entries comprise negative amounts that have the opposite effect of the original entry.

Examples 1 and 2 describe functionality of the COA Module 102. Example 1: An employee entered a punch, which was approved by their supervisor, for 4 hours work from 8 am to 12 pm on June 2^{nd}. The employee later corrects the punch to indicate they worked for 5 hours from 8 am to 1 pm on June 2^{nd}. To correct the amount, a compensating entry may be created using the COA Module 102 reducing the employee account balance by 4 hours, and a separate entry may be created increasing the employee account balance by 4 hours. The two new entries may refer to the previous/original entry and be pending until approved by the employee's supervisor. FIG. 19 illustrates an original entry, a compensating entry, and a replacement entry as described herein.

### Example 2

An employee entered a punch, which was approved by their supervisor, for 4 hours work from 8 am to 12 pm on June 2^{nd}. The supervisor later determines the employee did not work that day and cancels the punch. To correct the amount, a compensating entry may be created using the COA Module 102 reducing the employee account balance by 4 hours. A new entry will refer to the previous entry and will be pending until approved by the employee's supervisor. FIG. 20 illustrates an original entry, and a compensating entry as described herein.

In some embodiments, the Payroll Module 216 may include functionality to determine overtime pay by: determining the start and end of the pay week, based on the start date/time of the punch, sum the processed punch entries for the employee for the pay week, and determine the portion of the punch that is overtime. The Payroll Module 216 may determine holiday pay by: determining if the date of a punch is a holiday in the employee's holiday schedule, and determining if the employee has a holiday account.

A payroll batch process which may be implemented using the Payroll Module 216 is provided in Table 1 below:

TABLE 1

| Line | Pseudocode | Description |
|---|---|---|
| 1 | With Batch_Criteria SET EmployeeList to | Execute a database query |
| | Select List of Distinct Employees | to return a distinct list of |
| | | Employee profiles that are |
| | | effected by the payroll batch |
| | | operation based on |
| | | batching criteria. Criteria |
| | | could include: all unpaid |
| | | entries prior to a defined |
| | | end date, payroll entries for |
| | | employees with a specific |
| | | default cost center, or even |
| | | entries for a specific |
| | | employee. |
| 2 | FOREACH Employee in EmployeeList | Generate the payroll entries |
| | | for each employee in the |
| | | employee list. |
| 3 | | |
| 4 | SET EntryList to Select List of Entries for | Execute a database query |
| | Employee where Entry.Status == Approved and | to return unpaid punch |
| | Entry.StartDate < EndPayrollDate | entries for the current |
| | | employee in the list. Entries |
| | | should have a start date |
| | | prior to the specified end |
| | | payroll date for the batch |
| | | (pay period). |
| 5 | FOREACH Entry in EntryList | Generate payroll entries for |
| | | each punch entry for the |
| | | current employee. |
| 6 | SET PayWeek = GetPayWeek(Entry) | Call a function to determine |
| | | the pay week for the current |
| | | punch entry. |
| 7 | SET Hours PaidToDate = | Call a function to determine |
| | GetHoursPaidToDate(PayWeek) | the hours that have been |
| | | paid for the current |
| | | employee for the current |
| | | pay week. |
| 8 | SET EntryHoursPaidToDate = | Call a function to determine |
| | GetEntryHoursToDate(Entry) | the number of hours that |
| | | have already been paid of |
| | | the current punch entry. |
| | | Some entries may span pay |
| | | periods; in which case only |
| | | the portion of the punch that |
| | | falls prior to the end payroll |
| | | date will be processed. |
| 9 | SET EntryHours = Entry.Hours − | Determine how many hours |
| | EntryHoursPaidToDate | of the punch entry remain to |
| | | be paid. |
| 10 | SWITCH Employee.Type | Process the payroll for the |
| | | punch entry based on the |
| | | employee type. |
| 11 | CASE NonExempt: // Salary | Process non-exempt |
| | | (salary) employee. |
| 12 | PayEntry(Entry, Entry.Amount, | Call a function to generate a |
| | PayType.Regular) | payroll entry as regular time |
| 13 | END CASE | |
| 14 | CASE Exempt: // Hourly | Process exempt (hourly) |
| | | employee. |
| 15 | SET OvertimeHours = HoursPaidToDate + | Determine and process the |
| | EntryHours − | appropriate number of |
| | Settings.RegularTimeHours | regular and overtime hours. |
| | IF OvertimeHours > 0 THEN | Overtime is processed first |
| | PayEntry(Entry, OverTimeHours, | and deducted from the |
| | PayType.Overtime) | hours to be paid for the |
| | EntryHours = EntryHours − | entry. Call a function to |
| | OvertimeHours | generate a payroll entry for |
| | ELSE | overtime. Call a function to |
| | OvertimeHours = 0 | generate a payroll entry for |
| | END IF | regular time. |
| | IF EntryHours > 0 THEN | |
| | PayEntry(Entry, EntryHours, | |
| | PayType.Regular) | |
| | END IF | |
| 16 | END CASE // Hourly | |
| 17 | END SWITCH // Employee.Type | |
| 19 | END FOREACH // EntryList | |
| 20 | | |
| 21 | END FOREACH // EmployeeList | |

In some embodiments, the computing platform 100 includes extensive attachment, note, and event capture functionalities. Attachments can be ‘pinned’ to any action recorded in the computing platform 100, and the computing platform 100 supports MIME format attachments. Examples of supported attachment file types include, but are not limited to: .pdf, .doc, .docx, xls, .xslx, .tiff, .jpg, .png, .avi, .mp3, and .mp4.

Attachment functionality may allow Participants, Employees, and other users to upload documents, pictures, and videos to appropriate locations throughout the computing platform 100. This allows users to share information about their care needs with their Employees. For example, a Participant can upload a video to their client profile that demonstrates how to properly lift and transfer them from a wheelchair into a vehicle. This video can be viewed by their Employee(s) at any time. An additional great use-case for attachment functionality is for person-centered plans created and uploaded to the computing platform 100. These plans can then be accessed by the Participant and anyone on their support team.

In addition, notes can be created for any system record or event (e.g. punch, account, authorization, user, etc.) in the computing platform 100. Notes can be categorized by type and note subject, and both the note and note body of text are enabled to accept attachments. For example, an Employee can upload a note about a service delivered to a Participant, and in the body of the note the Employee can upload a picture of work that was performed. “Canned notes” may also be implemented as provided by the funding source. For example, for a particular service code such as HAB, the computing platform 100 can be configured to have multiple “canned note” check boxes that the Employee can be required to select before closing a punch. Canned note checkboxes allow for easy analytic reporting and tracking for common required tasks associated with service codes such as: “hygiene”, “housekeeping”, or “medication administration.”

The computing platform 100 may extensively logs transactions and activity for auditing and investigation purposes. The computing platform 100 captures events on all entities within the system, and stores this data in a user-friendly format that can be viewed by those with appropriate roles. The computing platform 100 may store the following information for all captured events:
Date and time of event;
Subject for event (e.g., Creation, View, Update/Edit, Approval, Verification, Log In/Out, etc.);
Description; and
User.

Returning to FIG. 3, implementation and further detail of the computing platform 100 may be illustrated referencing a process flow 300. Referring to block 302 of process flow 300, an account as described herein may be generated. The account may define a relationship between an Employee, a client, a service code, and a funding source. More particularly for example, the account may define that the Employee is authorized to provide a service to the client associated with a service code X, and that the funding source has been deemed responsible for compensating the client based on a service instance associated with the service code X.

Referring to block 304, as described herein, the Employee may be geo-located during travel to the client location, and also while at the location of the client to verify the Employee's physical location relative to the client.

Referring to block 306, EVV may be conducted to provide another layer of verification that the Employee is actually with the client performing a service instance associated with the authorized service code X. This EVV may include facial recognition where the Employee or client takes a picture of the client as further described herein, or the client provides other electronic verification that the Employee is present with the client.

Referring to block 308, as described herein, assuming EVV is successful and the service instance is otherwise approved and verified, an electronic transaction may be recorded to reflect the time entry submitted by the Employee.

Referring to block 310, the application 116 may further be leveraged or otherwise configured to transmit a request or otherwise request compensation from the funding source associated with the account created in block 302. The service code X is referenced to ensure that compensation is processed effectively.

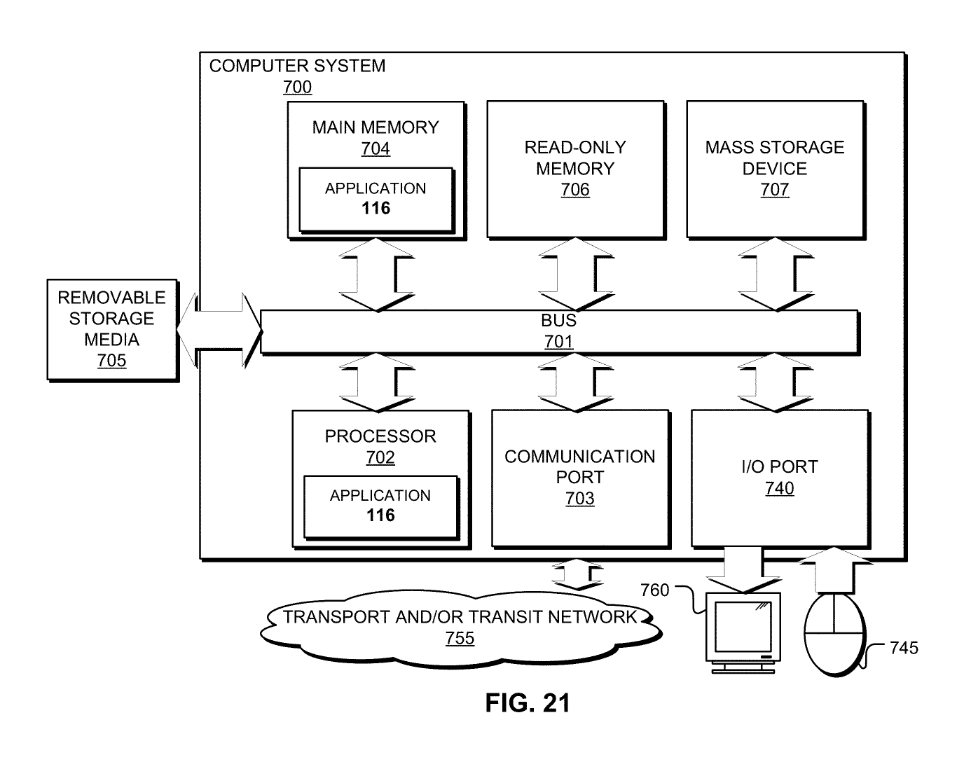

FIG. 21 is an example schematic diagram of a computing system 700 that may implement various methodologies discussed herein. For example, the computing system 700 may comprise a server used to execute aspects of the care management application 116 including the COA Module 102. The computing system 700 includes a bus 701 (i.e., interconnect), at least one processor 702 or other computing element, at least one communication port 703, a main memory 704, a removable storage media 705, a read-only memory 706, and a mass storage device 707. Processor(s) 702 can be any known processor, such as, but not limited to, an Intel® Itanium® or Itanium 2® processor(s), AMD® Opteron® or Athlon MP® processor(s), or Motorola® lines of processors. Communication port 703 can be any of an RS-232 port for use with a modem based dial-up connection, a 10/100 Ethernet port, a Gigabit port using copper or fiber, or a USB port. Communication port(s) 703 may be chosen depending on a network such as a Local Area Network (LAN), a Wide Area Network (WAN), or any network to which the computer system 700 connects. Computing system may further include a transport and/or transit network 755, a display screen 760, an I/O port 740, and an input device 745 such as a mouse or keyboard.

Main memory 704 can be Random Access Memory (RAM) or any other dynamic storage device(s) commonly known in the art. Read-only memory 706 can be any static storage device(s) such as Programmable Read-Only Memory (PROM) chips for storing static information such as instructions for processor 702. Mass storage device 707 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices, may be used.

Bus 701 communicatively couples processor(s) 702 with the other memory, storage, and communications blocks. Bus 701 can be a PCI/PCI-X, SCSI, or Universal Serial Bus (USB) based system bus (or other) depending on the storage devices used. Removable storage media 705 can be any kind of external hard drives, thumb drives, Compact Disc-Read Only Memory (CD-ROM), Compact Disc-Re-Writable (CD-RW), Digital Video Disk-Read Only Memory (DVD-ROM), etc.

Embodiments herein may be provided as a computer program product, which may include a machine-readable medium having stored thereon instructions which may be used to program a computer (or other electronic devices) to perform a process. The machine-readable medium may include, but is not limited to optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

As shown, main memory 704 may be encoded with the application 116 that supports functionality discussed above. In other words, aspects of the application 116 (and/or other resources as described herein) can be embodied as software code such as data and/or logic instructions (e.g., code stored in the memory or on another computer readable medium such as a disk) that supports processing functionality according to different embodiments described herein. During operation of one embodiment, processor(s) 702 accesses main memory 704 via the use of bus 701 in order to launch, run, execute, interpret or otherwise perform processes, such as through logic instructions, executing on the processor 702 and based on the application 116 stored in main memory or otherwise tangibly stored.

The description above includes example systems, methods, techniques, instruction sequences, and/or computer program products that embody techniques of the present disclosure. However, it is understood that the described disclosure may be practiced without these specific details. In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to optical storage medium (e.g., CD-ROM); magneto-optical storage medium, read only memory (ROM); random access memory (RAM); erasable programmable memory (e.g., EPROM and EEPROM); flash memory; or other types of medium suitable for storing electronic instructions.

Certain embodiments are described herein as including one or more modules, e.g. care modules 104. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” or “module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure a processor, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

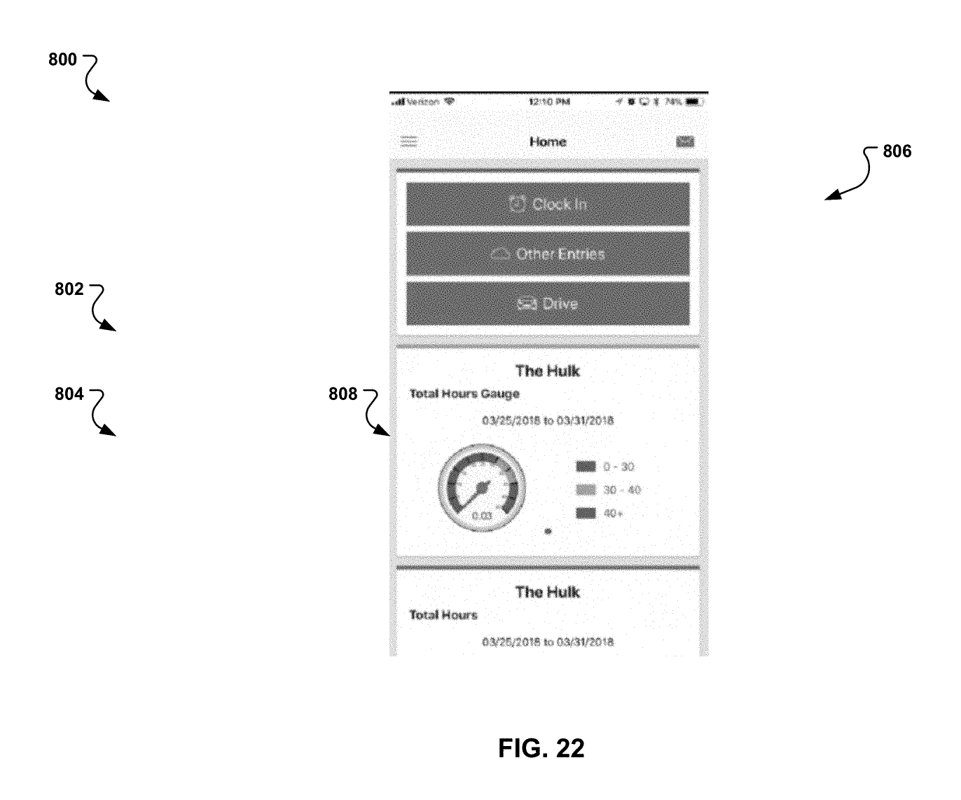

Referring to FIGS. 22-25, as discussed above, the care management application 116 is configured to accommodate multiple modes of electronic visit verification, or EVV, so that confirmation can be provided that an employer has visited a client to provide some form of care service. As shown in these figures, aspects of the care management application 116 including EVV may be provided via a mobile device 800 (such as a smartphone, tablet, or other such mobile device) executing a mobile application 802 comprising a graphical user interface (GUI) 804, which may be implemented by the Employee as he/she is providing a care service to the client or patient.

Referring to FIG. 22, a screen shot 806 of the mobile application 802 is shown, illustrating a home page of the GUI 804. In this example, the Employee logs in to the mobile application 802, and may clock-in (if the Employee is already at the location of a client or otherwise), or set a Drive (predetermined Employee drive module to track Employee travel) location to track the Employee's travel to and from the client, as further described herein. As further shown, the mobile application 802 may be configured with a plurality of widgets 808 which may graphically display, e.g., a total number of hours worked by the Employee, and messaging functionality, among other information. The home screen shown is configurable to illustrate aspects of training, driving, hourly service, and other aspects of care service and may include messaging functionality (not shown) to accommodate communications e.g., between the client and the Employee. In some embodiments, all or at least most of the information handled by the mobile application 802 is not stored locally. In other words, all of the information managed or accessed by the mobile application 802 may be stored on server infrastructure and/or the SaaS/PaaS environment 108 to increase security of the overall

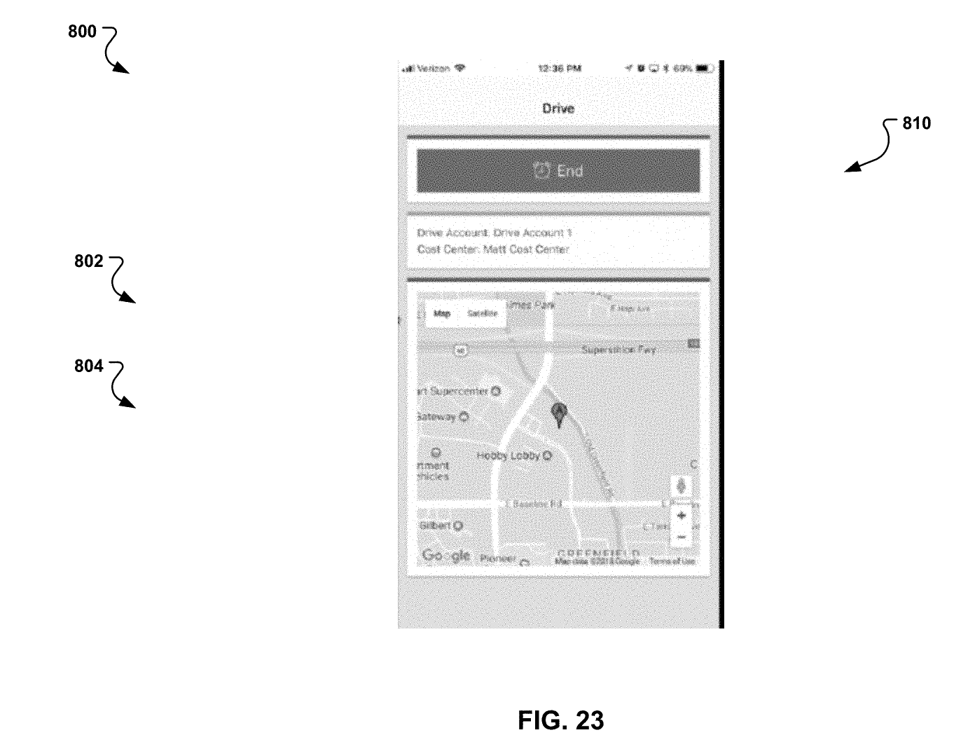

Referring to FIG. 23, additional information regarding the Drive functionality and map configuration is shown by a screen shot 810 of the mobile application 802. In some embodiments, the mobile application 802 may be configured with functionality and data provided by e.g., Google Maps, by calling one or more APIs associated with Google Maps or otherwise integrating functionality and information provided thereof. Utilizing functionality of Google Maps accommodates the ability to geo-locate an Employee for EVV purposes. In one example, a trip may be configured or specified for an Employee which defines an expected time it should take from an Employee to reach a given client. In other words, this expected time, which can be closely estimated using Google Maps or other functionality, can provide a baseline time it should take for the Employee to reach the client to provide a care service. A deviation or variance parameter may be assigned or set within the mobile application 802 to account for possible delays associated with the Employee as her/she travels to and from the client. For example, where the baseline time to and from a client is 30 minutes, a deviation parameter of 10 minutes may be assigned to provide the Employee with some cushion of time to anticipate natural delays during travel. Where an Employee exceeds the baseline time and the variance (e.g., takes over 40 min to meet the patient) an administrator may be electronically notified.

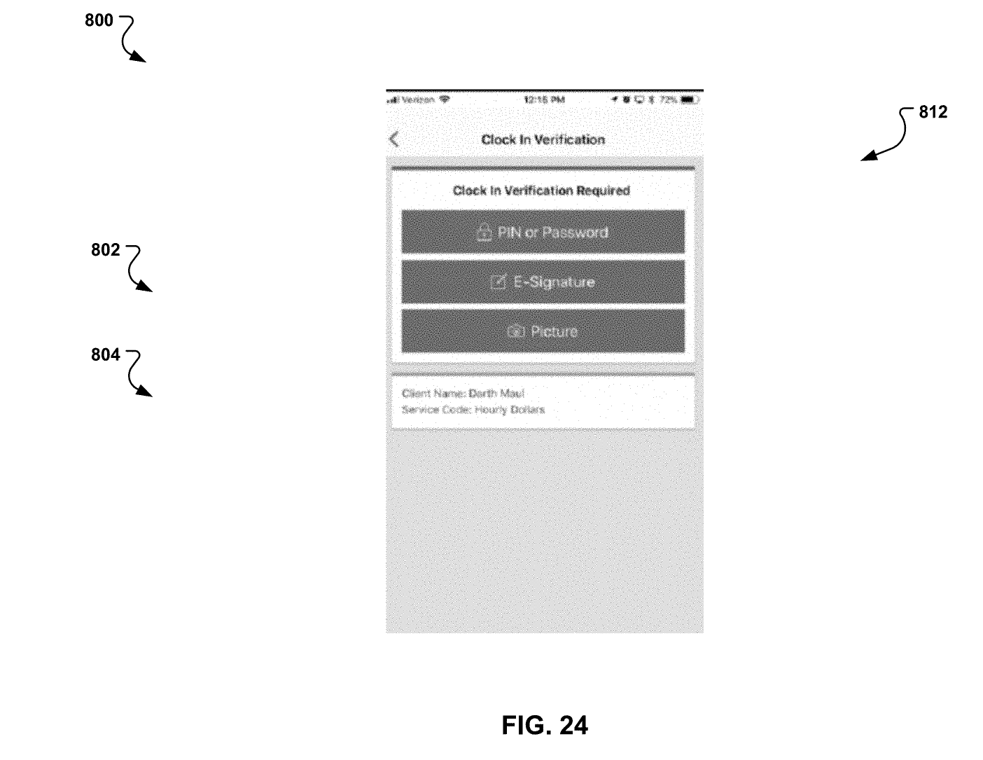

Referring to FIG. 24, a screen shot 812 is shown for illustrating the EVV described herein, shown in the screen shot 812 as “Clock-in Verification.” EVV can be configured for implementation at clock-in, at clock-out, during predetermined intervals as an Employee as visiting a client, or combinations thereof. When conducting EVV at a predetermine interval or time during an Employee visit with a client, the mobile application 802 may display a notification (not shown) requesting EVV during a predetermined time period.

As shown, EVV may be conducted using a variety of electronic methods. For example, the client may be requested to provide a PIN or password to indicate that the Employee is in fact present with the client and providing a care service. Alternatively, or in addition, the client may provide an electronic signature for electronic verification of the visit.

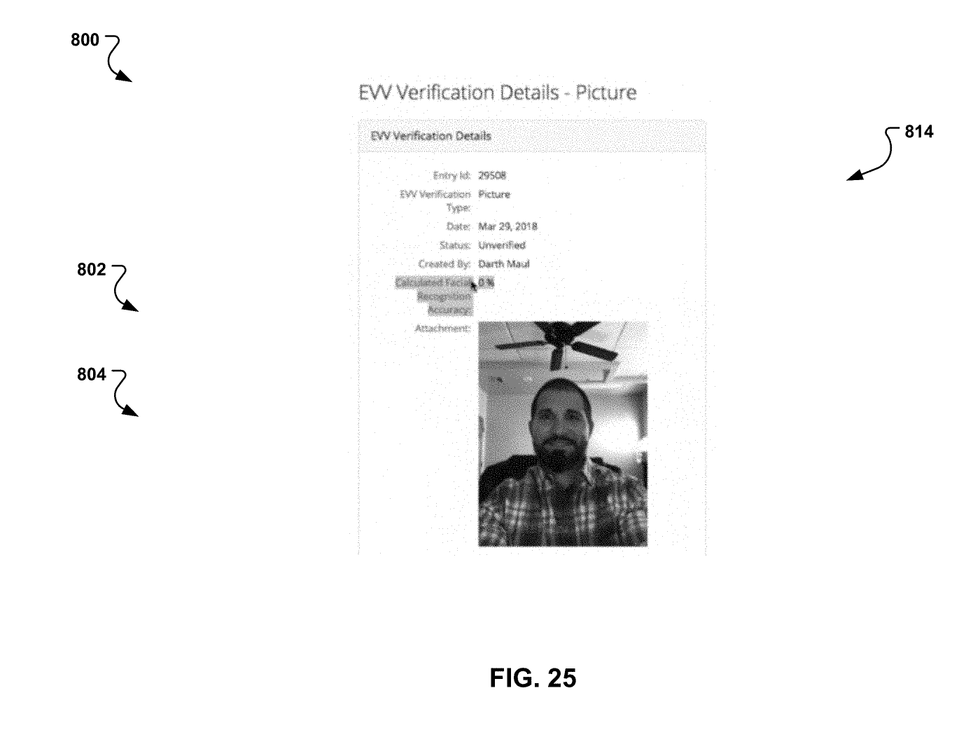

Referring to FIGS. 24-25, including screen shot 812 of FIG. 24 and screen shot 814 of FIG. 25, EVV may also be conducted using a digital photograph or picture and facial recognition as described herein. Specifically, a photograph may be taken of a client, to create a reference picture. Features of the reference picture may be extracted or otherwise processed using a facial recognition application, such as the facial recognition functionality provided by Microsoft Azure, and the features of the reference picture may be stored in memory for subsequent use as needed.

When an Employee clocks in using the EVV functionality, the mobile application 802 may require the client to acknowledge the Employee's presence using the picture EVV function, or the Employee may simply decide to use the same. Specifically, a camera (not shown) of the mobile device 800 may be used to take a new picture of the client at or around clock-in by the Employee. The mobile application 802 is configured to process this new picture, extract a set of features from the new picture, compare the features of the new picture to features of the reference picture, and generate a value, such as an accuracy percentage, which may be used to indicate whether the new picture actually represents the client. In addition, a threshold percentage may be assigned to the client which may be predetermined based upon the client, or Employee or otherwise. For example, in one embodiment, the threshold percentage may be 90%. In this example, EVV may be deemed successful where an accuracy percentage is generated that meets or exceeds the threshold percentage of 90%. Providing a configurable accuracy threshold may allow some deviation to consider changes to the client or other factors such as changes in hair style, clothing, facial hair, poor lighting, and the like. It should be understood that multiple new pictures may be generated to reflect a plurality of visits to a client by an Employee. The facial recognition functionality utilizes machine learning such that the more pictures that are processed using the methods described herein, the more accurate the EVV facial recognition becomes.

Once the new picture is generated for comparison with the reference picture, the new picture may be time-stamped, and information about geo-location may also be included or overlaid along the new picture. The ability of the care management application 116 and the mobile application 802 to geo-locate an Employee and client, and conduct facial recognition EVV is believed to be a considerable improvement in the art by providing dual-verification that the caregiver Employee is actually on site providing a service, and that this service is acknowledged by the client/patient. Each new picture and EVV implementation may be correlated or tied to a punch or record of an Employee visit with a client.

In addition, although not shown, the mobile application 802 may include functionality to allow a client to provide an Employee with a rating for a visit, i.e., a caregiver rating. This caregiver rating may be applied to an Employee record, and may be updated to reflect additional visits or services provided by the Employee.

It is believed that the present disclosure and many of its attendant advantages should be understood by the foregoing description, and it should be apparent that various changes may be made in the form, construction, and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it should be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A method, comprising:
utilizing a processor in communication with a tangible storage medium storing instructions that are executed by the processor to perform operations comprising:
generating an account, the account defining a plurality of entities associated with care management for a first healthcare provider;
defining a relationship between a patient and an employee associated with the plurality of entities;
assigning a service code to the account, the service code defining a type of care service the employee is authorized to provide to the patient by the first healthcare provider; and
assigning a funding source to the account.

2. The method of claim 1, further comprising:
accessing a time entry associated with the employee of the account; and
generating a transaction defining a debit and a credit associated with the time entry.

3. The method of claim 1, further comprising generating an additional account defining a plurality of entities associated with care management for a second healthcare provider.

4. The method of claim 2, further comprising:
verifying that the employee provided the care service to the patient reflected by the time entry; and
verifying that the time entry is associated with the service code to determine whether the employee provided an authorized service.

5. The method of claim 4, wherein the verifying that the employee provided the care service to the patient reflected by the time entry comprises electronically analyzing a set of geolocation coordinates and a phone number associated with the patient.

6. The method of claim 4, wherein verifying that the employee provided the care service to the patient reflected by the time entry comprises receiving a confirmation from a client or guardian associated with the client via a client device.

7. The method of claim 4, wherein verifying that the employee provided the care service to the patient reflected by the time entry comprises:
providing a FOB to the patient; and
receiving confirmation via the FOB that the Employee has generated a time punch with two tokens associated with the time punch, a first token of the two tokens defining a first time associated with entry by the Employee, and a second token of the two tokens defining a second time associated with exit by the employee, wherein the difference between the first time and the second time satisfies a predetermined deviation value.

8. The method of claim 2, further comprising:
modifying the time entry, by
generating a compensating time entry to modify a time value associated with the time entry, and
generating a replacement entry.
